(19) United States
(12) Patent Application Publication
(10) Pub. No.: US 2021/0145451 A1
(43) Pub. Date: May 20, 2021

(54) HEMOSTATIC DEVICE

(71) Applicant: TERUMO KABUSHIKI KAISHA, Shibuya-ku (JP)

(72) Inventors: Fumi WATANABE, Hadano (JP); Masako MIYASHITA, Fujinomiya (JP); Yuichi HIOKI, Fuji (JP); Ryo OKAMURA, Fujinomiya (JP); Satoshi WADA, Fujinomiya (JP)

(73) Assignee: TERUMO KABUSHIKI KAISHA, Shibuya-ku (JP)

(21) Appl. No.: 17/160,865

(22) Filed: Jan. 28, 2021

Related U.S. Application Data
(63) Continuation of application No. PCT/JP2019/029820, filed on Jul. 30, 2019.

(30) Foreign Application Priority Data
Aug. 1, 2018 (JP) 2018-145366

(51) Int. Cl. A61B 17/135; A61B 17/132

(52) U.S. Cl. CPC A61B 17/135; A61B 2017/12004; A61B 17/1325

(57) ABSTRACT

A hemostatic device is comprised of a covering member that includes a main body to which an inflatable member is connected, a first arm portion extending away from the main body, and a second arm portion extending away from the main body while forming an obtuse angle with a longitudinal direction of the first arm portion. The first arm portion includes an enlarged portion protruding in a width direction of the first arm portion at an end portion of the first arm portion. The second arm portion is longer than the first arm portion and is configured to be secured to the enlarged portion and to be secured to the second arm portion by passing between fingers of the patient in a state in which the second arm portion is wrapped around a limb of a patient.

### CROSS-REFERENCES TO RELATED APPLICATIONS

This application is a continuation of International Application No. PCT/JP2019/029820 filed on Jul. 30, 2019, which claims priority to Japanese Patent Application No. 2018-145366 filed on Aug. 1, 2018, the entire content of both of which is incorporated herein by reference.

### FIELD OF THE INVENTION

The present invention generally relates to a hemostatic device and method of performing hemostasis.

### BACKGROUND DISCUSSION

As a catheter procedure, a procedure is known in which a blood vessel (for example, radial artery) in an arm of a patient is punctured, and various medical elongated bodies are introduced into the blood vessel via a puncture site formed in the blood vessel in the arm of the patient so as to perform treatment or therapy on a lesion area. An example of this is disclosed in Japanese Patent Application Publication No. 2008-119517. The catheter procedure utilizing the radial artery is referred to as transradial artery approach and is considered a useful technique for, for example, coronary artery access and lower limb artery access.

A radial artery located in an arm of a human body is connected to a palmar artery which bypasses a hand side. Therefore, currently, as a new method of transradial artery approach, a catheter procedure using distal transradial approach (dTRA) has been attempted to access the palmar artery (including a distal radial artery) from an anatomical snuffbox located on a dorsal side of the hand or from a position around the snuffbox, and perform treatment through the vascular access site.

### SUMMARY

Blood vessels such as palmar arteries located in the hand are located in places where there are many movable parts such as fingers. For this reason, a shape around the puncture site in the hand changes with movement of the hand. Therefore, when hemostasis is performed on the puncture site, a pressing member placed on the hand is preferably an inflatable member that follows the movement of the hand and can easily adjust a compressive force on the puncture site.

However, when the pressing member is the inflatable member, the inflatable member exerts a force to inflate from the inside to the outside of the inflatable member in an inflated state. For this reason, in a hemostatic device having the inflatable member, a shape around a hemostatic site changes due to the movement of the hand, the inflatable member is shifted from the puncture site, and it may not be possible to properly maintain the compressive force of the inflatable member on the puncture site in some cases. Therefore, when hemostasis is performed on the puncture site of the hand, the hemostatic device having the inflatable member needs to suppress position shift of the inflatable member by force acting in a direction away from the puncture site, thereby appropriately securing the inflatable member to the puncture site. In this way, it is considered that the hemostatic device having the inflatable member can appropriately maintain the compressive force of the inflatable member on the puncture site even when the shape around the puncture site is changed by the movement of the hand.

The hemostatic device disclosed here is capable of suppressing position shift of an inflatable member with respect to a site where bleeding is to be stopped on a hand and appropriately maintaining a compressive force of the inflatable member on the site where bleeding is to be stopped on the hand even when a patient moves the hand while the inflatable member is inflated.

A hemostatic device according to one aspect includes a covering member configured to cover a site on a hand of a patient where bleeding is to be stopped, a securing member connected to the covering member and configured to secure the covering member on the hand of the patient while the covering member is covering the site on the patient's hand where bleeding is to be stopped, and an inflatable member connected to the covering member and configured to be inflated by injection of a fluid. The covering member includes a main body to which the inflatable member is connected, a first arm portion extending away from the main body in a longitudinal direction of extent, and a second arm portion extending away from the main body in a direction forming an obtuse angle with the longitudinal direction of extent of the first arm portion. The first arm portion includes one end portion closest to the main body and an opposite end portion remote from the main body, with the first arm portion including an enlarged portion that protrudes in a width direction of the first arm portion at the opposite end portion of the first arm portion. The second arm portion is longer than the first arm portion, wherein the second arm portion includes a first part configured to be secured to the enlarged portion by way of a first part of the securing member. The second arm portion is configured to pass between fingers of the hand of the patient while the second arm portion is wrapped around a limb of the patient and includes a second part configured to be secured to a third part of the second arm portion by way of a second part of the securing member.

A hemostatic device according to another aspect includes a covering member configured to cover a site on a hand of a patient where bleeding is to be stopped, a securing member connected to the covering member and configured to secure the covering member on the hand of the patient while the covering member is covering the site on the patient's hand where bleeding is to be stopped, and an inflatable member connected to the covering member and configured to be inflated by injection of a fluid. The covering member includes a main body to which the inflatable member is connected, a first arm portion extending away from the main body in a longitudinal direction of extent, and a second arm portion extending away from the main body in a direction forming an obtuse angle with the longitudinal direction of extent of the first arm portion. The inflatable member includes a first inflatable portion and a deformable auxiliary member which has a smaller outer shape than an outer shape of the first inflatable portion and is offset toward one end side of the first inflatable portion. The first arm portion and the second arm portion of the covering member are configured to be connectable to one another by the securing member in a state in which the covering member is wrapped around a limb of the patient. The auxiliary member is disposed between the first arm portion and the second arm portion.

The hemostatic device is configured so that the main body in which the inflatable member is disposed can be secured to the limb by disposing a part of the second arm portion between adjacent fingers of the patient and connecting the enlarged portion and the second arm portion while wrapping the first arm portion and the second arm portion around the limb of the patient. The first arm portion and the second arm portion are fastened to the limb by connecting the enlarged portion and the second arm portion, and the main body is secured to the limb. Further, by disposing the second arm portion between the adjacent fingers of the patient, it is possible to suppress position shift of the inflatable member disposed in the main body with respect to the site where bleeding is to be stopped. Further, the hemostatic device has a simple securing structure capable of tightening the inflatable member to the hand of the patient by connecting the two arm portions protruding from the main body. For this reason, the hemostatic device can reduce the number of arm portions installed for securing the inflatable member. In this way, the hemostatic device can suppress an increase in arm portions that may interfere with a medical device such as an introducer in a state where the hemostatic device is attached to the hand of the patient, and thus it is possible to easily remove the medical device from the site where bleeding is to be stopped even after the hemostatic device is attached. In addition, a movable range of the hand increases from a wrist side to a fingertip side of the hand. In the hemostatic device, the first arm portion and the second arm portion extend from the fingertip side to the wrist side of the hand while forming an obtuse angle, so that the first arm portion and the second arm portion can be connected on the wrist side of the hand of the patient. Therefore, in the hemostatic device, the first arm portion and the second arm portion can be secured on the wrist side where the movable range is small, and thus the main body can be secured to the limb while maintaining the movable range on the fingertip side of the hand. An outer circumference of the hand becomes larger from the wrist side to the fingertip side of the hand in a state where the hand is spread. Therefore, since the first arm portion and the second arm portion extend in a direction opposite to a direction in which the outer circumference of the hand becomes larger, the first arm portion and the second arm portion can be reliably connected on the wrist side of the hand in the state where the hemostatic device is attached. Furthermore, in the hemostatic device, even in the case where the patient moves the hand with the inflatable member inflated, when the first arm portion and the second arm portion are secured in a state of being tightened to the limb of the patient, it is possible to prevent rising of the distal side (fingertip side) of the main body to which the inflatable member is connected, and to appropriately maintain the compressive force of the inflatable member on the site where bleeding is to be stopped formed on the dorsal side of the hand of the patient.

In addition, since the first arm portion and the second arm portion extend from the main body so as to form an obtuse angle, when the hemostatic device is attached to the limb of the patient, the respective arm portions can be connected so that the entire finger of the patient is not covered by the covering member. For this reason, the patient is less likely to be restricted in movement of the hand even when the hemostatic device is attached, and thus a degree of freedom on the fingertip side can be increased. Further, since the hemostatic device has the deformable auxiliary member located on the distal side of the first inflatable portion, when the first inflatable portion is inflated, the auxiliary member presses the first inflatable portion against the limb of the patient to inhibit the first inflatable portion from rising in a direction away from the puncture site on the hand of the patient. As a result, the hemostatic device can maintain an appropriate compressive force on the site where bleeding is to be stopped while increasing the degree of freedom on the fingertip side. Furthermore, in the hemostatic device, since the first arm portion and the second arm portion extend from the fingertip side to the wrist side of the hand while forming an obtuse angle, the first arm portion and the second arm portion can be secured on the wrist side where the movable range is small. Therefore, the main body can be appropriately secured to the limb while maintaining the movable range on the fingertip side of the hand. Further, in the hemostatic device, since the first inflatable portion can be secured to the site where bleeding is to be stopped by the two arm portions including the first arm portion and the second arm portion and the auxiliary member, it is unnecessary to add different arm portions between the first arm portion and the second arm portion. Therefore, the operator, etc. can easily remove the medical device such as an introducer sheath after attaching the hemostatic device. Further, since the first arm portion and the second arm portion are connected in a state of being wrapped around the limb of the patient, the first arm portion and the second arm portion are pressed against the hand of the patient while pulling the main body located therebetween toward the both side portion sides of the main body. Since the auxiliary member is located between the first arm portion and the second arm portion, the inflatable member can be reliably pressed against the body surface of the hand of the patient by connection of the first arm portion and the second arm portion. Therefore, the hemostatic device can press the first inflatable portion against the limb of the patient by the auxiliary member, and reliably inhibit the first inflatable portion from rising in the direction away from the site where bleeding is to be stopped on the hand of the patient. In this way, the hemostatic device can reliably press the first inflatable portion against the hand of the patient and maintain an appropriate compressive force on the puncture site while increasing the degree of freedom on the fingertip side during attachment of the hemostatic device.

Another aspect involves a method of producing hemostasis at a puncture site on a hand of a patient where bleeding is to be stopped. The method involves positioning a main body of a covering member in overlying relation to the puncture site on the hand of the patient where bleeding is to be stopped so that an inflatable member underlying the main body portion is positioned relative to the puncture site. The covering member also includes a first arm portion extending away from the main body in a longitudinal direction of extent, and a second arm portion extending away from the main body in a direction forming an obtuse angle with the longitudinal direction of extent of the first arm portion. The first arm portion includes one end portion closest to the main body and an opposite end portion remote from the main body, with the opposite end portion of the first arm portion including an enlarged portion that protrudes in a width direction of the first arm portion, and the second arm portion being longer than the first arm portion. The method additionally involves wrapping the first arm portion and the second arm portion around a limb of the patient while the main body is in overlying relation to the puncture site on the hand of the patient, securing a first part of the second arm portion and the enlarged portion of the first arm to one another while the first part of the second arm portion is in contact with the enlarged portion of the first arm, passing the second arm portion between two fingers of the hand of the patient, securing a second part of the second arm portion and a third part of the second arm portion to one another while the second part of the second arm portion and the third part of the second arm portion are in contact with one another, and introducing a fluid into the inflatable member to inflate the inflatable member and apply a compressive force from the inflatable member to the puncture site to perform hemostasis.

### BRIEF DESCRIPTION OF THE DRAWINGS

FIG. 1 is a diagram illustrating a hemostatic device according to a first embodiment, and is a plan view seen from an outer surface side of a main body of a covering member.

FIG. 2 is a diagram illustrating the hemostatic device according to the first embodiment, and is a plan view seen from an inner surface side of the main body of the covering member.

FIG. 3 is an enlarged view of a part of the hemostatic device.

FIG. 4 is a cross-sectional view of the hemostatic device taken along the section line 4-4 in FIG. 3, and a diagram illustrating a state in which an inflatable member inflates.

FIG. 5A is a diagram schematically illustrating a usage example of the hemostatic device according to the first embodiment.

FIG. 5B is a diagram schematically illustrating a usage example of the hemostatic device according to the first embodiment.

FIG. 5C is a diagram schematically illustrating a usage example of the hemostatic device according to the first embodiment.

FIG. 5D is a diagram schematically illustrating a usage example of the hemostatic device according to the first embodiment.

FIG. 5E is a diagram schematically illustrating a usage example of the hemostatic device according to the first embodiment.

FIG. 6 is a diagram schematically illustrating a part of a cross section taken along the section line 6-6 in FIG. 5E.

FIG. 7A is a diagram schematically illustrating another usage example of the hemostatic device according to the first embodiment.

FIG. 7B is a diagram schematically illustrating another usage example of the hemostatic device according to the first embodiment.

FIG. 7C is a diagram schematically illustrating another usage example of the hemostatic device according to the first embodiment.

FIG. 7D is a diagram schematically illustrating another usage example of the hemostatic device according to the first embodiment.

FIG. 8 is a plan view of the hemostatic device according to a modification of the first embodiment.

FIG. 9 is a cross-sectional view of the hemostatic device taken along the section line 9-9 in FIG. 8.

FIG. 10 is a diagram illustrating a hemostatic device according to a second embodiment, and is a plan view seen from an outer surface side of a main body of a covering member.

FIG. 11 is a diagram illustrating the hemostatic device according to the second embodiment, and is a plan view seen from an inner surface side of the main body of the covering member.

FIG. 12A is a diagram schematically illustrating a usage example of the hemostatic device according to the second embodiment.

FIG. 12B is a diagram schematically illustrating a usage example of the hemostatic device according to the second embodiment.

### DETAILED DESCRIPTION

Set forth below with reference to the accompanying drawings is a detailed description of embodiments of a hemostatic device and method of performing hemostasis representing examples of the inventive hemostatic device and method disclosed here. The dimensions or scales on the drawings may be exaggerated or different from actuality/reality for convenience of description and illustration. The following description does not limit the technical scope or the meaning of terms in the appended claims.

### First Embodiment

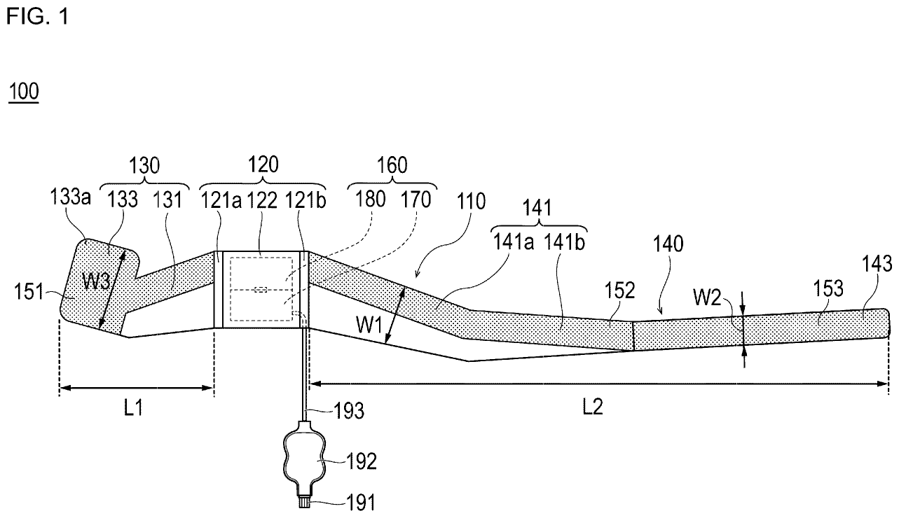

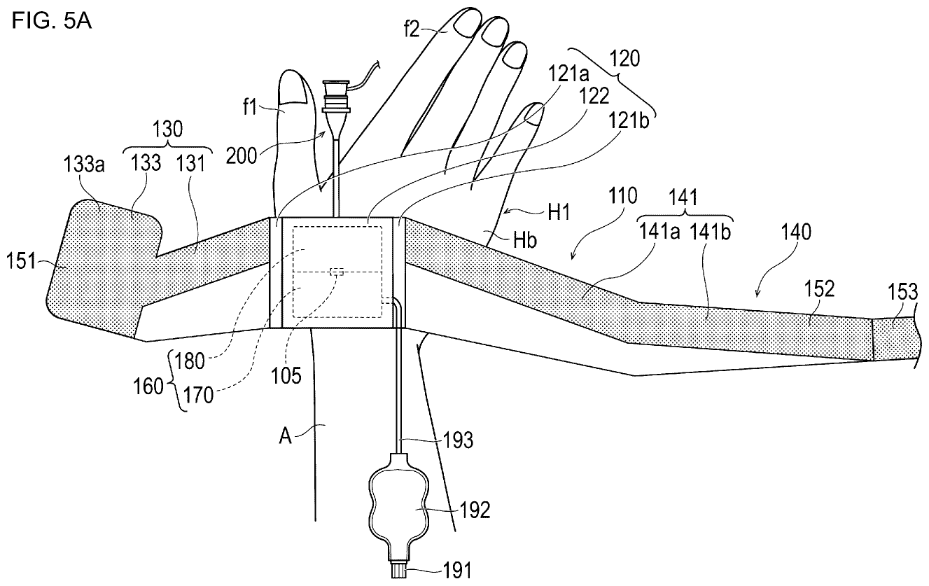

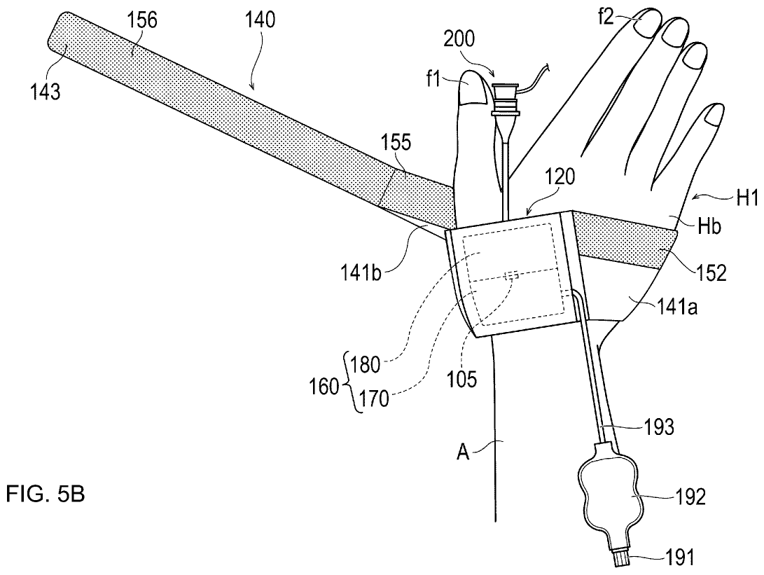

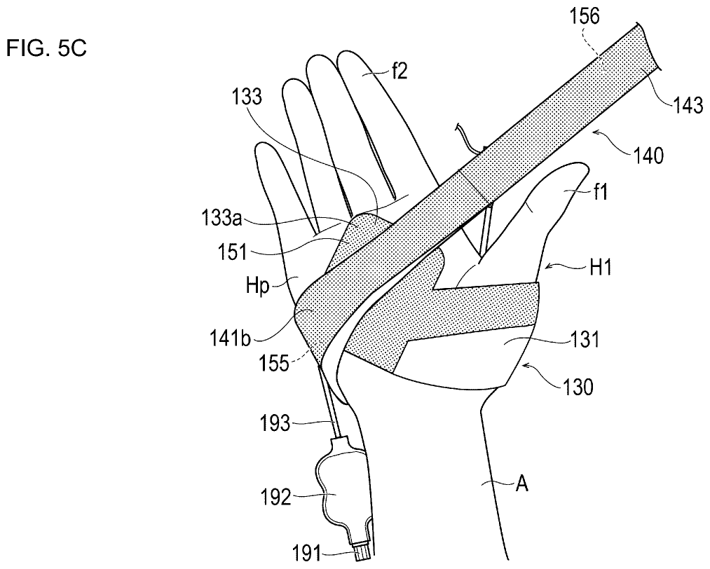

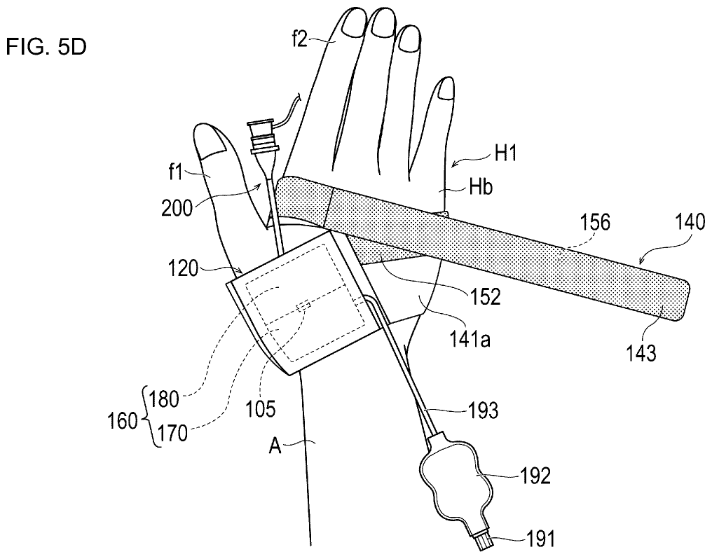

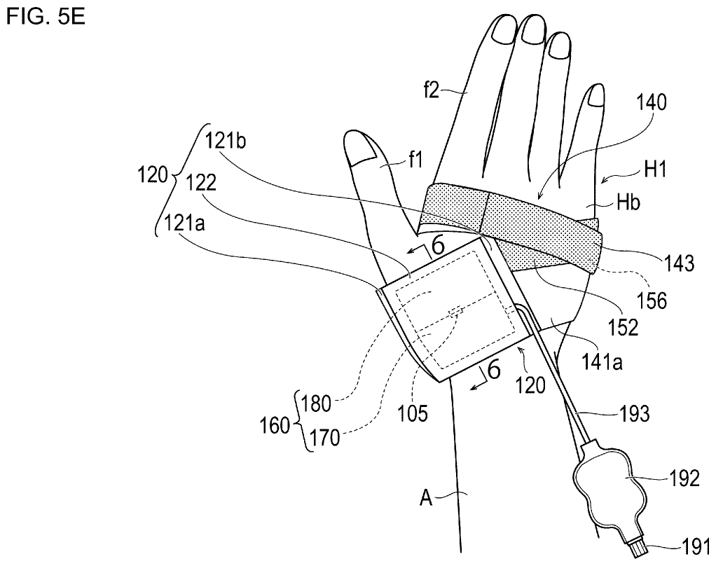

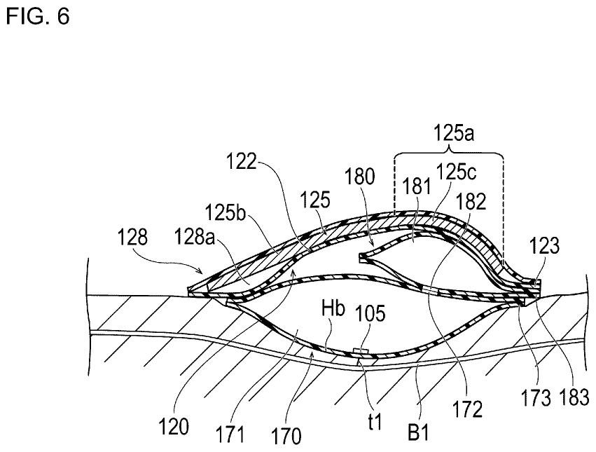

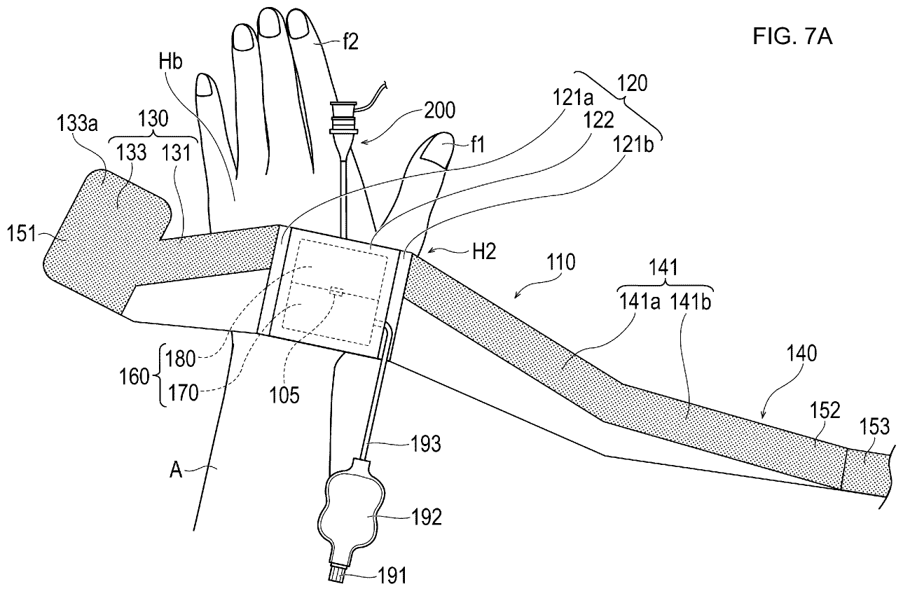

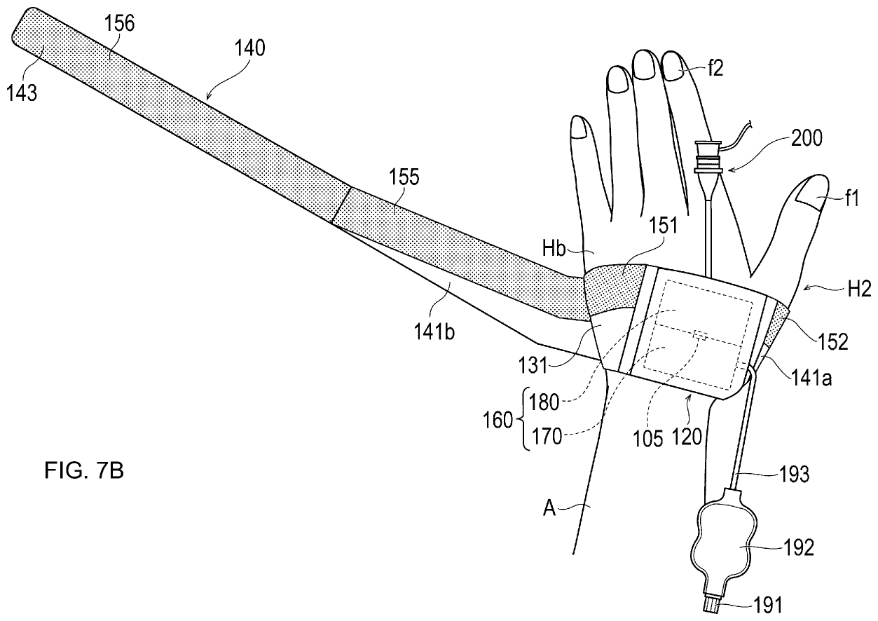

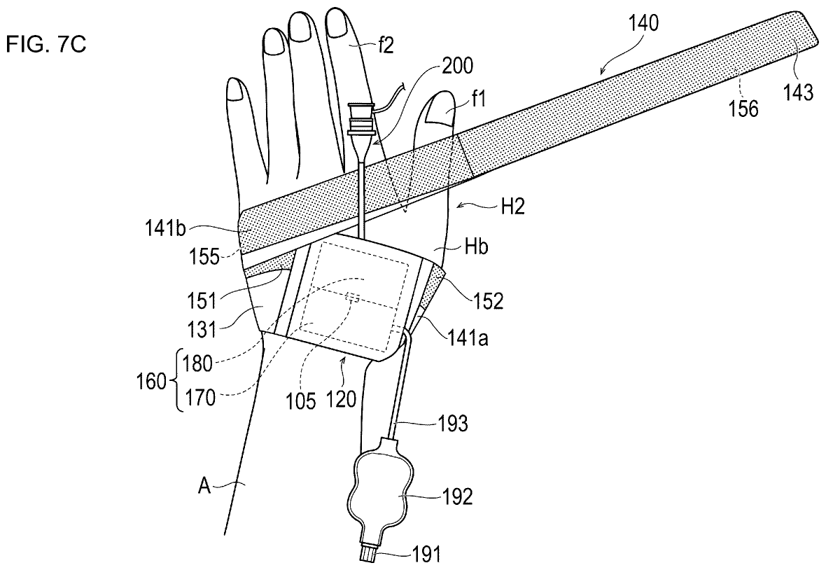

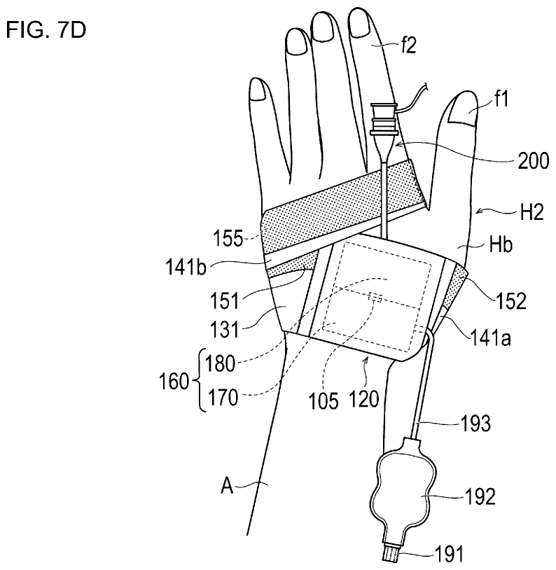

FIGS. 1 to 4 are diagrams for description of a hemostatic device 100 according to a first embodiment, FIGS. 5A to 6 are diagrams for description of usage examples of the hemostatic device 100, and FIGS. 7A to 7D are diagrams for description of other usage examples of the hemostatic device 100.

As illustrated in FIGS. 5E, 6, and 7D, when removing, for example, a sheath tube of an introducer 200 (shown in FIG. 5A) indwelling at a puncture site t1 (corresponding to a site where bleeding is to be stopped) formed on a radial artery side (for example, an artery around an anatomical snuffbox or a distal radial artery running on a fingertip side of the snuffbox) of a palmar artery (deep palmar artery) B1 running on or extending along a dorsal side Hb of a right hand H1 (or a left hand H2) located on the fingertip side of a forearm A of a patient, the hemostatic device 100 is used to perform hemostasis on the puncture site t1. The anatomical snuffbox is a cavity in the hand located on the radial side of the forearm A when the patient spreads a thumb f1 of the right hand H1 or the left hand H2.

In brief, as illustrated in FIGS. 1, 2, and 6, the hemostatic device 100 includes a covering member 110 configured to be disposed to cover the puncture site t1 on the hand H1 (right hand in this example) of the patient, a plurality of securing members 151, 152, 153, 154, 155, and 156 for securing the covering member 110 while the covering member 110 covers the puncture site t1, and an inflatable member 160 connected to the covering member 110 and configured to be inflated by injection of a fluid.

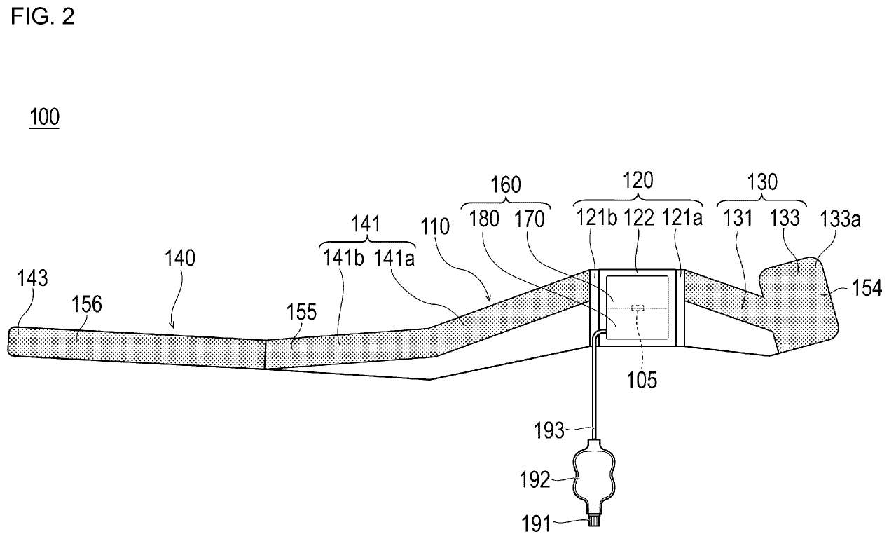

FIG. 1 illustrates a plan view of the hemostatic device 100 seen from an outer surface side of a main body 120 of the covering member 110, and FIG. 2 illustrates a plan view of the hemostatic device 100 seen from an inner surface side of the main body 120 of the covering member 110. The inner surface side of the main body 120 is a surface on a side to which the inflatable member 160 disposed to face a body surface of the patient when the hemostatic device 100 is attached to the patient is connected, and the outer surface side of the main body 120 is a surface opposite to the inner surface (surface on a side where a support member 125 is disposed in the present embodiment). In addition, a “distal side” used in the following description is a side on which the fingertip(s) of the right hand H1 is disposed (upper side in FIGS. 1 and 5E) in a state in which hemostatic device 100 is attached to the right hand H1 of the patient.

<Covering Member>

As illustrated in FIGS. 1 and 2, the covering member 110 includes the main body 120 to which the inflatable member 160 is connected, a first arm portion 130 protruding or extending from the main body 120, and a second arm portion 140 protruding or extending from the main body 120 while forming an obtuse angle with a longitudinal direction of the first arm portion 130.

The first arm portion 130 has an inclined portion 131 located on an end portion side connected to a first region 121a of the main body 120, and an enlarged portion 133a protruding in a width direction of the first arm portion 130 at an end portion 133 opposite to a side where the inclined portion 131 is disposed. In the illustrated embodiment, the enlarged portion 133a is positioned at the free end of the first arm portion 130 disposed remote from the main body 120 as shown in FIGS. 1 and 2.

As illustrated in FIGS. 1 and 2, the longitudinal direction of the first arm portion 130 is a left-right direction in FIGS. 1 and 2 in which the first arm portion 130 extends in a state in which the first arm portion 130 is extended without attaching (i.e., before attaching) the hemostatic device 100 to the right hand H1 of the patient. In addition, a width direction of the first arm portion 130 is a direction intersecting the longitudinal direction of the first arm portion 130 on the plan views illustrated in FIGS. 1 and 2 and an up-down (vertical) direction in FIGS. 1 and 2. Similarly, a longitudinal direction of the second arm portion 140 is the left-right direction in FIGS. 1 and 2 in which the second arm portion 140 extends in a state in which the second arm portion 140 is extended without attaching (i.e., before attaching) the hemostatic device 100 to the right hand H1 of the patient, and a width direction of the second arm portion 140 is the up-down (vertical) direction of FIGS. 1 and 2 intersecting the longitudinal direction of the second arm portion 140 in the plan views illustrated in FIGS. 1 and 2.

The inclined portion 131 and the enlarged portion 133a of the first arm portion 130 are formed of a flexible band-shaped member that allows the first arm portion 130 to be wrapped around the right hand H1 of the patient.

As illustrated in FIG. 1, the inclined portion 131 is inclined in a direction away from a distal side of the main body 120. The enlarged portion 133a protrudes toward the distal side of the main body 120 in a direction intersecting the longitudinal direction of the first arm portion 130. That is, the enlarged portion 133a protrudes toward the distal direction (upper direction) in FIGS. 1 and 2. Further, the enlarged portion 133a is inclined in a direction toward the main body 120 side. A width W3 of the enlarged portion 133a may be 25 mm to 40 mm.

As illustrated in FIGS. 1 and 2, the second arm portion 140 is longer than the first arm portion 130. Further, as illustrated in FIGS. 5B, 5C, and 5D, the second arm portion 140 is configured to be secured to the enlarged portion 133a of the first arm portion 130 and secured to the second arm portion 140 by passing between fingers f1 and f2 of the patient in a state in which the second arm portion 140 is wrapped around a limb of the patient.

The second arm portion 140 is formed of a flexible band-shaped member that can be wrapped around the limb of the patient. The limb around which the second arm portion 140 is wrapped includes, for example, a part of the right hand H1 of the patient, a part of the forearm A, and a part of a wrist. Further, a space between the fingers (inter-finger portion) through which the second arm portion 140 passes is, for example, a space between the thumb f1 and the index finger f2 of the right hand H1 of the patient. However, a position of the limb around which the second arm portion 140 is wrapped or a position of the inter-finger portion through which the second arm portion 140 is passed is not particularly limited. The hemostatic device 100 according to the present embodiment can be attached to the left hand H2 in the same manner as each part of the right hand H1 described above (see FIG. 7D).

As illustrated in FIG. 1, a magnitude relationship between a length L1 of the first arm portion 130 and a length L2 of the second arm portion 140 (i.e., the length L1 of the first arm portion 130 relative to the length L2 of the second arm portion 140) can be defined by lineal distances L1 and L2 of the respective arm portions 130 and 140 in a state in which the respective arm portions 130 and 140 are extended and before the hemostatic device 100 is attached to the right hand H1 of the patient (i.e., in the plan views shown in FIGS. 1 and 2). By way of example, the length L1 of the first arm portion 130 may be 80 mm to 400 mm. In addition, the length L2 of the second arm portion 140 may be, for example, 350 mm to 400 mm.

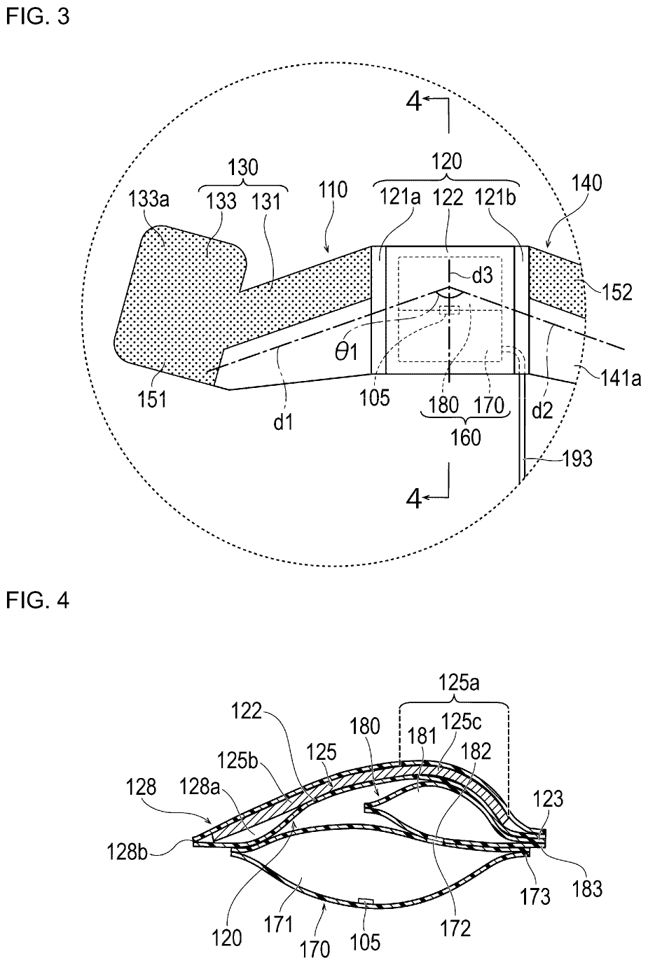

In the present embodiment, as illustrated in FIG. 3, an obtuse angle θ1 between the longitudinal direction of the first arm portion 130 and the longitudinal direction of the second arm portion 140 can be defined as an angle formed by the intersection of a straight line d1 extending substantially parallel to a longitudinal direction of the inclined portion 131 of the first arm portion 130 and a straight line d2 extending substantially parallel to a longitudinal direction of a first inclined portion 141a of the second arm portion 140 on the main body 120. By way of example, the obtuse angle θ1 may be 120° to 170°.

As described above, in the hemostatic device 100, the first arm portion 130 and the second arm portion 140 form an obtuse angle on the plan views illustrated in FIGS. 1 and 3. Therefore, the first arm portion 130 is inclined and extends in a direction away from the distal side of the main body 120. Similarly, the second arm portion 140 is inclined and extends in a direction away from the distal side of the main body 120. Therefore, the first arm portion 130 and the second arm portion 140 extend from the distal side of the main body 120 toward the proximal side so as to spread in the shape of the kanji character for the number eight without intersecting each other. That is, the first arm portion 130 and the second arm portion 140 extend from the distal side of the main body 120 toward the proximal side so as to spread in the shape of an inverted V-shape (i.e., A) without intersecting each other.

An angle formed by a straight line d3 (vertical line in FIG. 3) passing through a center of the main body 120 (corresponding to a center line of a first inflatable portion 170) and a straight line d1 along the longitudinal direction of the first arm portion 130 may be the same as or different from an angle formed by the straight line d3 passing through the center of the main body 120 and a straight line d2 along the longitudinal direction of the second arm portion 140.

As illustrated in FIGS. 1 and 2, the second arm portion 140 has an inclined portion 141 and a second arm end portion 143 that is continuously formed with the inclined portion 141 and forms an end portion of the second arm portion 140.

The inclined portion 141 of the second arm portion 140 includes the first inclined portion 141a connected to the first region 121a of the main body 120 and a second inclined portion 141b extending between the first inclined portion 141a and the second arm end portion 143.

The first inclined portion 141a is inclined in a direction away from the distal side of the main body 120. Similarly to the first inclined portion 141a, the second inclined portion 141b is inclined in a direction away from the distal side of the main body 120. As shown in FIG. 1, the second inclined portion 141b may be inclined relative to the first inclined portion 141a.

A width of the first inclined portion 141a gradually decreases from the first region 121b side of the main body 120 toward the second inclined portion 141b side. A width of the second inclined portion 141b gradually decreases from the first inclined portion 141a side to the second arm end portion 143 side.

The second arm end portion 143 extends linearly along an extending direction of the second arm portion 140 with a substantially constant width. The second arm end portion 143 is a portion including a predetermined range in the extending direction of the second arm portion 140 from an end portion (terminal) of the second arm portion 140.

As illustrated in FIG. 1, a width W1 of the inclined portion 141 is larger than a width W2 of the second arm end portion 143. The width W1 is a width of the inclined portion (the first inclined portion 141a and the second inclined portion 141b) 140 at any position along the inclined portion.

By way of example, the width W1 of the inclined portion 141 may be 15 mm to 45 mm. Further, for example, the width W2 of the second arm end portion 143 may be 10 mm to 20 mm. A maximum value of the width W1 of the inclined portion 141 illustrated above is a maximum value of the first inclined portion 141a, and a minimum value of the width W1 of the inclined portion 141 is a minimum value of the second inclined portion 141b. The inclined portion 141 can be formed by one inclined portion extending from the first region 121b side of the main body 120 to the second arm end portion 143. Even in such a configuration, the width W1 of the inclined portion 141 discussed above may still apply.

In the first arm portion 130 and the second arm portion 140, for example, it is possible to provide an entry portion (information entry portion) that allows the operator, etc. to enter information such as the amount of air injected into the inflatable member 160, a hemostatic time, etc. in a procedure using the hemostatic device 100. The entry portion can be made of a material allowing the information, etc. to be written using ink, etc. provided in a known pen.

As illustrated in FIG. 3, the main body 120 of the covering member 110 includes the first regions 121a and 121b, and a second region 122 in which the inflatable member 160 is disposed unlike the first regions 121a and 121b. That is, the inflatable member 160 is disposed at the second region 122, and is not disposed at the first regions 121a and 121b. The second region 122 is thus disposed at a location different from the locations of the first regions 121a and 121b.

The first region 121a is disposed between the second region 122 and the first arm portion 130. The first region 121b is disposed between the second region 122 and the second arm portion 140.

As illustrated in FIG. 4, the second region 122 of the main body 120 includes the support member 125 having a greater rigidity than that of the first regions 121a and 121b of the main body 120 (i.e., the support member 125 has a greater rigidity than that of the first regions 121a and 121b and so the main body in the second region 122 is more rigid than the main body in the first regions 121a and 121b). In the support member 125, the side portion sides located in the left-right direction are adjacent to the first regions 121a and 121b on the plan view illustrated in FIG. 3.

As illustrated in FIG. 4, the support member 125 is inserted into or positioned in an insertion portion 128a disposed on an outer surface side of the second region 122 (surface opposite to a side where each of the inflatable portions 170 and 180 is disposed in the second region 122, which is a surface on an upper side of FIG. 4).

The insertion portion 128a is a space formed between the cover member 128 disposed to cover a part of the outer surface of the main body 120 and the main body 120.

An insertion port 128b communicating with the insertion portion 128a is formed on the proximal side (lower side of FIG. 3 and left side of FIG. 4) of the main body 120. The support member 125 can be inserted into the insertion portion 128a via the insertion port 128b.

The cover member 128 can be connected to the covering member 110 by, for example, adhesion or welding. In the present embodiment, the cover member 128 is connected to the covering member 110 at three sides, namely the three sides other than a part where the insertion port 128b is formed.

As illustrated in FIG. 4, the support member 125 has a curved portion 125a formed on the distal side of the main body 120. That is, the support member 125 has a longitudinal extent extending between one end portion closer to the distal side of the main body 120 and an opposite end portion remote from the distal side of the main body 120, and the one end portion of the support member closer to the distal side of the main body 120 includes the curved portion 125a. The curved portion 125a has a cross-sectional shape protruding or curved upward so as to be separated or spaced from the main body 120 on the cross-sectional view illustrated in FIG. 4. Stated differently, the curved portion 125a includes a concave surface (downward facing surface in FIG. 4) facing towards the inflatable member 160 and a concave surface (upward facing surface in FIG. 4) facing away from the inflatable member 160. The distal side of the man body 120 refers to an upper side of the center position with reference to a center position (position where a marker portion 105 is disposed in the present embodiment) on the plan view of the inflatable member 160 illustrated in FIG. 3.

When the inflatable member 160 is inflated, the curved portion 125a of the support member 125 wraps or extends around (encircles) the vicinity of the end portion of the inflatable member 160 on the distal side (an end portion 173 of the first inflatable portion 170 and an end portion 183 of the second inflatable portion 180), so that the compressive force of the inflatable member 160 is directed toward the central portion of the support member 125 (center portion in the left-right direction of FIG. 4). Thus, the compressive force of the inflatable member 160 is inhibited from escaping to the outside of the support member 125. As a result, the hemostatic device 100 can suppress a decrease in the compressive force of the inflatable member 160 on the puncture site t1, and thus it is possible to appropriately maintain the compressive force of the inflatable member 160 on the puncture site t1.

An apex 125c of the curved portion 125a of the support member 125 (a part of the curved portion 125a most distant from the inflatable member 160) can be disposed, for example, on the distal side of the main body 120 with respect to the center position of the inflatable member 160. That is, the apex may be displaced away from the center of the inflatable member 160 and toward the distal side of the inflatable member 160. By disposing the apex 125c of the curved portion 125a at the position described above, the support member 125 can reliably wrap or encircle the vicinity of the end portion of the inflatable member 160 on the distal side when the inflatable member 160 inflates, and thus it is possible to effectively inhibit the compressive force of the inflatable member 160 from escaping to the outside of the support member 125.

As illustrated in FIG. 4, the support member 125 has an inclined portion 125b extending from the curved portion 125a toward the proximal side of the main body 120. The inclined portion 125b extends substantially linearly toward the proximal side of the main body 120.

A width direction dimension (vertical direction dimension of FIG. 3) of the support member 125 is not particularly limited. However, it is preferable to adopt a size that, for example, allows the proximal end of the support member 125 to be disposed near the proximal end of the main body 120 in a state in which a distal end of the support member 125 is disposed near a distal end of the auxiliary member 180 as illustrated in FIG. 4. Further, a longitudinal direction dimension (left-right direction dimension of FIG. 3) of the support member 125 is not particularly limited. However, when the first regions 121a and 121b are formed on the main body 120, it is preferable to, for example, adopt a dimension that allows formation of the first regions 121a and 121b having desired sizes on both side portion sides of the main body 120.

The support member 125 may be disposed, for example, on the inner surface side of the main body 120 of the covering member 110 (surface on a side where each of the inflatable portions 170 and 180 is disposed, which is a surface on a lower side of FIG. 4). When the support member 125 is disposed in this way, connection of the cover member 128 to the main body 120 can be omitted, and each of the inflatable portions 170 and 180 can be fixed to the support member 125.

In the present embodiment, the first regions 121a and 121b and the second region 122 of the main body 120 are integrally formed of one member to form a unitary, one-piece member as shown. However, the first region 121a and 121b and the second region 122 may be configured by connecting different or separate members.

In the second region 122 of the main body 120, the cover member 128, the support member 125, the first inflatable portion 170, and the second inflatable portion 180, a portion overlapping the marker portion 105 in the plan view illustrated in FIGS. 1 and 3 and surroundings thereof are preferably transparent (including colored transparent, colorless transparent, and translucent). By adopting such a configuration, the operator can visually recognize the puncture site t1 from the outer surface side of the main body 120 even when the marker portion 105 is superposed on the puncture site t1.

It is preferable that the first regions 121a and 121b of the main body 120 are made of a material having higher elasticity than that of the first arm portion 130 and the second arm portion 140. In this way, when the hemostatic device 100 is attached to the right hand H1 of the patient, by wrapping each of the arm portions 130 and 140 around the limb of the patient, the first region 121a is pulled toward the first arm portion 130 side and extended, and the first region 121b is pulled toward the second arm portion 140 side and extended. As a result, a physical property of the main body 120 changes at each boundary between the first arm portion 130 and the second arm portion 140 to allow the main body to be deformed relatively easily, and thus the first arm portion 130 and the second arm portion 140 can be easily disposed on the right hand H1 of the patient in a state where the main body 120 is disposed on the puncture site t1 formed on the right hand H1 of the patient.

Further, a material of the support member 125 is preferably more rigid than a material of each of the first arm portion 130 and the second arm portion 140. As a result, when the inflatable member 160 inflates, the support member 125 can suppress rising of the main body 120 by the inflatable member 160 due to the rigidity of the support member 125. Further, since the first arm portion 130 and the second arm portion 140 are configured to be more flexible than the support member 125, the hemostatic device 100 can be easily attached to the patient along the limb of the patient when being attached to the patient.

In the present embodiment, in the covering member 110, each of the main body 120, the first arm portion 130, and the second arm portion 140 is formed as a separate member. When each portion of the covering member 110 is configured as a separate member in this way, each of the main body 120, the first arm portion 130, and the second arm portion 140 can be connected by, for example, adhesion, welding, etc. However, in the covering member 110, any of the main body 120, the first arm portion 130, and the second arm portion 140 may be integrally formed as one member.

The material used to fabricate the main body 120 of the covering member 110 is not particularly limited. Examples of such material include polyvinyl chloride, polyolefin such as polyethylene, polypropylene, polybutadiene, or ethylene-vinyl acetate copolymer (EVA), polyester such as polyethylene terephthalate (PET) or polybutylene terephthalate (PBT), polyvinylidene chloride, silicone, polyurethane, various thermoplastic elastomers such polyamide elastomer, polyurethane elastomer, and polyester elastomer, nylon, nylon elastomer, or any combination thereof (blended resin, polymer alloy, laminate, etc.).

The material used to fabricate the cover member 128 is not particularly limited, and examples of such material include the same materials as those exemplified as the material of the covering member 110.

The material used to fabricate the support member 125 preferably has a higher rigidity than that of the material used for the first regions 121a and 121b of the main body 120 of the covering member 110. Examples of such a material may include acrylic resin, polyvinyl chloride (especially hard polyvinyl chloride), polyolefin such as polyethylene, polypropylene, or polybutadiene, polystyrene, poly-(4-methylpentene-1), polycarbonate, ABS resin, polymethylmethacrylate (PMMA), polyacetal, polyacrylate, polyacrylonitrile, polyvinylidene fluoride, ionomer, acrylonitrile-butadiene-styrene copolymer, polyester such as polyethylene terephthalate (PET) or polybutylene terephthalate (PBT), butadiene-styrene copolymer, aromatic or aliphatic polyamide, fluorine-based resin such as polytetrafluoroethylene, etc.

The material used to produce the first arm portion 130 and the second arm portion 140 of the covering member 110 is not particularly limited. Examples of such material may include the same materials as the materials described above to produce the main body 120 of the covering member 110, woven fabric, nonwoven fabric, felt, cloth, knitted fabric, and paper.

<Securing Member>

As illustrated in FIGS. 1 and 2, the hemostatic device 100 includes six securing members, namely a first securing member 151, a second securing member 152, a third securing member 153, a fourth securing member 154, a fifth securing member 155, and a sixth securing member 156.

As illustrated in FIG. 1, the first securing member 151 is disposed on the outer surface of the first arm portion 130. The first securing member 151 is disposed on a part of the inclined portion 131 on the distal side and on the entirety of the end portion 133.

As illustrated in FIG. 1, the second securing member 152 and the third securing member 153 are disposed on the outer surface of the second arm portion 140. The second securing member 152 is disposed on a part of the first inclined portion 141a on the distal side and on a part of the second inclined portion 141b on the distal side. The third securing member 153 is disposed on the entirety of the second arm end portion 143.

As illustrated in FIG. 2, the fourth securing member 154 is disposed on the inner surface of the first arm portion 130. The fourth securing member 154 is disposed on a part of the inclined portion 131 on the distal side and on the entirety of the end portion 133.

As illustrated in FIG. 2, the fifth securing member 155 and the sixth securing member 156 are disposed on the outer surface of the second arm portion 140. The fifth securing member 155 is disposed on a part of the first inclined portion 141a on the distal side and on a part of the second inclined portion 141b on the distal side. The sixth securing member 156 is disposed on the entirety of the second arm end portion 143.

The first securing member 151 and the second securing member 152 are formed on a male side of a surface fastener. The third securing member 153, the fourth securing member 154, the fifth securing member 155, and the sixth securing member 156 are formed on a female side of the surface fastener. The surface fastener is a fastener that is removable in terms of surface, and is, for example, MAGIC TAPE (registered trademark) or VELCRO (registered trademark).

A specific configuration of each of the securing members 151, 152, 153, 154, 155, and 156 is not limited as long as the second arm portion 140 wrapped around the limb of the patient can be secured to the enlarged portion 133a, and a part of the second arm portion 140 passed between the thumb f1 and the index finger f2 can be secured to the second arm portion 140. For example, some of the securing members 151, 152, 153, 154, 155, and 156 can be omitted, and positions where the securing members are disposed on the respective arm portions 130 and 140 can be changed as appropriate. Further, when each of the securing members 151, 152, 153, 154, 155, and 156 includes a surface fastener, a male side and a female side of the surface fastener may be interchanged. Further, for example, each of the securing members 151, 152, 153, 154, 155, and 156 may be a snap, a button, a clip, a frame member in which a hole is formed, etc.

<Inflatable Member>

As illustrated in FIGS. 3 and 4, the inflatable member 160 includes the first inflatable portion 170 and the deformable auxiliary member 180 having a smaller outer shape (outer shape on the plan view of FIG. 3) than that of the first inflatable portion 170.

The auxiliary member 180 overlaps the first inflatable portion 170 on the distal side of the main body 120.

The first inflatable portion 170 includes a lumen 171 into which a fluid can be injected, and a communication hole 172 formed at a position facing the auxiliary member 180.

In the present embodiment, the auxiliary member 180 is a second inflatable portion configured to be inflated by injection of a fluid. Hereinafter, the auxiliary member 180 will be referred to as a second inflatable portion.

The second inflatable portion 180 includes a lumen 181 into which a fluid can be injected, and a communication hole 182 disposed at a position facing the communication hole 172 of the first inflatable portion 170.

The lumen 181 of the second inflatable portion 180 communicates with the lumen 171 of the first inflatable portion 170 through the communication hole 182 of the second inflatable portion 180 and the communication hole 172 of the first inflatable portion 170.

As illustrated in FIG. 3, the second inflatable portion 180 is disposed to have bilateral symmetry with respect to the center line d3 of the first inflatable portion 170 (inflatable member 160). That is, the second inflatable portion 180 is symmetric about the line d3 and about a line perpendicular to the line d3 and passing through the center of the second inflatable portion 180. Further, the first securing member 151, the second securing member 152, and the third securing member 153 described above are disposed on the outer surface of the first arm portion 130 and the outer surface of the second arm portion 140 opposing to each other with the main body 120 interposed therebetween.

In the present embodiment, the second inflatable portion 180 has a substantially square shape. Therefore, in the second inflatable portion 180, a center position of the square in the left-right direction is disposed at the center position of the main body 120. Further, the marker portion 105, which will be described later, is disposed at the center of the second inflatable portion 180.

The second inflatable portion 180 is fixed to the inner surface of the covering member 110 (the inner surface of the main body 120 of the covering member 110). Specifically, the end portion 183 of the second inflatable portion 180 located on the distal side of the second inflatable portion 180 is fixed to the inner surface of the covering member 110.

In the first inflatable portion 170, the periphery of the communication hole 172 of the first inflatable portion 170 is fixed to the periphery of the communication hole 182 of the second inflatable portion 180. Specifically, in the first inflatable portion 170, only a certain range around each of the communication holes 172 and 182 located near the central portion of the second inflatable portion 180 is fixed to the second inflatable portion 180. As described above, the first inflatable portion 170 is not directly connected to the covering member 110, and is indirectly connected to the covering member 110 via the second inflatable portion 180. As a result, in the second inflatable portion 180, the peripheral edge of the second inflatable portion 180 is not fixed to the first inflatable portion 170, and thus the outer shape of the second inflatable portion 180 is freely deformed. For this reason, even when the patient bends the finger (for example, the thumb f1) or the wrist upward (to the dorsal side Hb of the hand), the hemostatic device 100 can maintain a large area in which the second inflatable portion 180 compresses the first inflatable portion 170. Therefore, the hemostatic device 100 can effectively apply a compressive force to the puncture site t1 from the inflatable member 160. In the first inflatable portion 170 and the second inflatable portion 180, for example, the first inflatable portion 170 and the second inflatable portion 180 may be integrally connected to the covering member 110 by fixing the end portion 183 of the second inflatable portion 180 to the covering member 110 in a state where the first inflatable portion 170 and the second inflatable portion 180 are connected.

The inflatable member 160 may be fixed to the inner surface of the covering member 110 at a position different from that shown in FIG. 3 and described above as long as the inflatable member 160 is fixed to the covering member 110 on the curved portion 125a side of the support member 125. Specifically, a part or the whole of the outer surface side of the second inflatable portion 180 may be fixed to the inner surface of the covering member 110 on the curved portion 125a side of the support member 125. Even in such a configuration, in the hemostatic device 100, a compression direction of the inflatable member 160 is directed to the center portion of the first inflatable portion 170 (the center portion in the left-right direction of FIG. 4) by the curved portion 125a of the support member 125. Therefore, the hemostatic device 100 suppresses a decrease in the compressive force due to the inflatable member 160 even when a gap is generated between the main body 120 and the body surface of the hand H due to movement of the finger of the patient or bending of the wrist.

Further, it is preferable that the second inflatable portion 180 is located inside the curved portion 125a of the support member 125 and overlaps the first inflatable portion 170. As a result, since the second inflatable portion 180 is located inside the curved portion 125a of the support member 125, the compressive force of the second inflatable portion 180 is directed toward the center portion of the support member 125 by the curved portion 125a, and inhibited from escaping to the outside of the support member 125. Further, when the second inflatable portion 180 overlaps the first inflatable portion 170 on the curved portion 125a side of the support member 125, the second inflatable portion 180 can prevent the first inflatable portion 170 from rising on the distal side of the right hand H1 or the left hand H2 while preventing position shift of the first inflatable portion 170. Therefore, the hemostatic device 100 can more reliably prevent the inflatable member 160 from rising, and can suppress a decrease in the compressive force applied to the puncture site t1 by the inflatable member 160.

The first inflatable portion 170 has a substantially square shape on the plan view illustrated in FIG. 2. The second inflatable portion 180 has a substantially rectangular shape that includes a set of long sides having substantially the same length as that of one side of the first inflatable portion 170 and a set of short sides having a length which is approximately half a length of one side of the first inflatable portion 170.

The lumen 171 of the first inflatable portion 170 communicates with a lumen of a tube 193 for supplying a fluid such as air to the first inflatable portion 170. As illustrated in FIGS. 1 and 3, the tube 193 is connected to the first inflatable portion 170 on the proximal side of the first inflatable portion 170. The tube 193 extends to the outside of the main body 120 through the inner surface side of the main body 120 of the covering member 110. A position where the tube 193 extends out from the first inflatable portion 170 is not particularly limited. However, as illustrated in FIG. 3, when the tube 193 extends out to the proximal side of the main body 120, so that the hemostatic device 100 is attached to the patient, the tube 193 is disposed laterally to the right hand H1 (in a direction intersecting a direction in which the finger of the right hand H1 extends) (see FIG. 5B). For this reason, when the hemostatic device 100 is attached to the patient, the tube 193 can be prevented from interfering with the introducer 200.

The tube 193 may be connected to the second inflatable portion 180. Further, a position at which the tube 193 extends to the outside of the main body 120 can be appropriately changed.

As illustrated in FIGS. 3 and 4, the hemostatic device 100 includes the marker portion (marker) 105 for aligning the inflatable member 160 with respect to the puncture site t1.

The marker portion 105 is disposed at a position corresponding to a substantially center position (center position with respect to the plan view illustrated in FIG. 2) of the first inflatable portion 170.

As illustrated in FIG. 4, for example, the marker portion 105 can be disposed on an inner surface of a side surface (inner surface) of the first inflatable portion 170 disposed to face the body surface. However, the marker portion 105 may be disposed, for example, on an internal surface of a surface (outer surface) opposite to the side surface of the first inflatable portion 170 disposed to face the body surface or an external surface of the first inflatable portion 170, an internal surface or an external surface of the main body 120 of the covering member 110, an internal surface or an external surface of the support member 125, an internal surface or an external surface of the cover member 128, etc. In addition, when a center portion of the first inflatable portion 170 and an end portion of the second inflatable portion 180 (an end portion on the proximal side, which is an end portion on the left side of FIG. 4) are disposed to overlap each other on the cross-sectional view illustrated in FIG. 4, the marker portion 105 may be disposed on an external surface of the end portion of the second inflatable portion 180.

The marker portion 105 preferably includes a transparent central portion and a colored linear frame portion surrounding the central portion. In this way, the operator can dispose or position the marker portion 105 at the puncture site t1 while confirming the puncture site t1 through the transparent central portion of the marker portion 105. For this reason, the operator can easily dispose the center position of the first inflatable portion 170 at the puncture site t1 using the marker portion 105. The marker portion 105 may be, for example, formed only by the colored central portion without having the frame portion. Further, a specific shape and color of the marker portion 105, a formation method on each portion of the hemostatic device 100, etc. are not particularly limited.

In the present embodiment, the first inflatable portion 170 is formed of two sheet-shaped members. For example, the first inflatable portion 170 can be formed by forming the lumen 171 between two sheet-shaped members each possessing a substantially rectangular shape and bonding outer peripheral edges of the two sheet-shaped members in this state. Similarly to the first inflatable portion 170, the second inflatable portion 180 can be formed of two substantially rectangular sheet-shaped members bonded together.

A method of bonding the sheet-shaped members forming the first inflatable portion 170, and a method of bonding the sheet-shaped members forming the second inflatable portion 180 are not particularly limited. For example, it is possible to use adhesion or welding. Further, a method of connecting the second inflatable portion 180 and the main body 120 of the covering member 110 is not particularly limited. For example, adhesion or welding can be used. Further, a method of fixing the first inflatable portion 170 and the second inflatable portion 180 is not particularly limited. For example, adhesion or welding can be adopted.

The first inflatable portion 170 and the second inflatable portion 180 may not have a structure in which plural sheet-shaped members are bonded together. The first inflatable portion 170 and the second inflatable portion 180 may be formed of, for example, one bag-shaped member in which a space into which a fluid can flow is formed.

A material used for producing the first inflatable portion 170 and the second inflatable portion 180 is not particularly limited, and examples of such material may include the same materials as those described above as the material of the main body 120 of the covering member 110.

<Injection Portion>

As illustrated in FIG. 1, the hemostatic device 100 includes an injection portion 191 for injecting a fluid into the inflatable member 160 (the first inflatable portion 170 and the second inflatable portion 180).

The injection portion 191 includes a connector having an incorporated check valve. A syringe can be connected to the injection portion 191.

A cushioning member 192 having an inflatable space is disposed between the injection portion 191 and the inflatable member 160. The cushioning member 192 includes a flexible bag-shaped member having a space formed inside. The cushioning member 192 may be provided with an arrow-shaped marker indicating a direction in which the syringe is inserted into the injection portion 191.

The injection portion 191 is connected to one end side of the cushioning member 192. A lumen of the injection portion 191 communicates with a space in the cushioning member 192. However, while the check valve incorporated in the injection portion 191 is closed, communication between the lumen of the injection portion 191 and the space in the cushioning member 192 is cut off.

A flexible tube 193 is connected to the other end of the cushioning member 192. A lumen of the tube 193 communicates with the space in the cushioning member 192. Further, in the tube 193, the other end portion opposite to one end portion connected to the cushioning member 192 is connected to the first inflatable portion 170. The lumen of the tube 193 communicates with the lumen 171 of the first inflatable portion 170.

The other end portion of the tube 193 can be connected to the first inflatable portion 170 using, for example, an adhesive, etc. while being interposed between the two sheet-shaped members forming the first inflatable portion 170. In the sheet-shaped members forming the first inflatable portion 170, for example, convex portions partially protruding outward from the sheet-shaped members may be formed at parts interposing the tube 193 therebetween.

To inflate the first inflatable portion 170 and the second inflatable portion 180, the operator inserts a distal tubular portion of a syringe into the injection portion 191 to open the check valve. The operator injects air in the syringe into the lumen 171 of the first inflatable portion 170 by pushing a plunger of the syringe with the check valve of the injection portion 191 open.

When air is injected into the lumen 171 of the first inflatable portion 170, the first inflatable portion 170 inflates. Further, the air injected into the lumen 171 of the first inflatable portion 170 flows into the lumen 181 of the second inflatable portion 180 via the communication hole 172 of the first inflatable portion 170 and the communication hole 182 of the second inflatable portion 180. When air flows into the lumen 181 of the second inflatable portion 180, the second inflatable portion 180 inflates. When the first inflatable portion 170 and the second inflatable portion 180 inflate, the cushioning member 192 communicating with the lumen 171 of the first inflatable portion 170 via the tube 193 inflates.

The space in cushioning member 192 and the lumen 171 of the first inflatable portion 170 are in communication with each other via the tube 193 at all times. The injection portion 191 maintains the check valve incorporated in the injection portion 191 in a closed state when the syringe is not inserted into the injection portion 191 to prevent air from leaking from the injection portion 191. For this reason, when the internal pressure of the inflatable member 160 increases due to movement of the right hand H of the patient, etc., the air in the lumen 171 of the first inflatable portion 170 and the lumen 181 of the second inflatable portion 180 moves to the cushioning member 192 side which is not pressed against the puncture site t1 by the covering member 110. In this way, the compressive force applied to the puncture site t1 by the inflatable member 160 is adjusted, and thus the compressive force to the puncture site t1 by the inflatable member 160 can be appropriately maintained.

When the operator contracts the first inflatable portion 170 and the second inflatable portion 180, the operator inserts the distal tubular portion of the syringe into the injection portion 191 and pulls the plunger of the syringe. By performing the above operation, the operator can discharge the air in the first inflatable portion 170 and the air in the second inflatable portion 180 to the syringe.

When the operator, etc. inflates the inflatable member 160, the operator, etc. can visually confirm that the first inflatable portion 170 and the second inflatable portion 180 can be pressurized without leakage of air by confirming expansion of the cushioning member 192.

Next, a usage example of the hemostatic device 100 will be described with reference to FIGS. 5A to 5E. In the following, a description will be given of an example of a procedure for attaching the hemostatic device 100 to the right hand H1 of the patient on which the puncture site t1 is formed.

FIG. 5A illustrates a state in which various procedures are performed by inserting the sheath tube of the introducer 200 into the distal radial artery side of the palmar artery B1 via the puncture site t1 (see FIG. 6) formed on the dorsal side Hb of the right hand H1 of the patient. Further, FIG. 5A illustrates a state in which a part of the sheath tube of the introducer 200 is pulled out from the puncture site t1 after the above procedure is completed.

At the start of hemostasis, as illustrated in FIG. 5A, the operator, etc. disposes the main body 120 of the covering member 110 so that the main body 120 overlaps a side of the dorsal side Hb of the right hand H1. In this instance, the marker portion 105 disposed at a substantially center position of the first inflatable portion 170 is disposed on the puncture site t1 (i.e., the marker 105 overlies the puncture site t1).

Subsequently, as illustrated in FIGS. 5B and 5C, the operator, etc. wraps the inclined portion (the first inclined portion 141a and the second inclined portion 141b) 141 of the second arm portion 140 along the right hand H1 of the patient while wrapping the enlarged portion 133a of the first arm portion 130 around the right hand H1 of the patient. In this instance, the operator, etc. secures the enlarged portion 133a and the second arm portion 140 via the respective securing members 151 and 155 by bringing the fifth securing member 155 (female side of the surface fastener) disposed on the inner surface of the inclined portion 141 into contact with the first securing member 151 (male side of the surface fastener) disposed on the outer surface of the enlarged portion 133a of the first arm portion 130 on the palm Hp side of the right hand H1 of the patient.

When the second arm portion 140 is secured to the enlarged portion 133a, the operator, etc. can confirm the position of the enlarged portion 133a and the shape of the enlarged portion 133a by the feel of the fingers. Therefore, the operator, etc. can guide the second arm portion 140 to the enlarged portion 133a disposed on the palm Hp side of the right hand H1 of the patient and recognize a direction in which the second arm portion 140 is disposed on the enlarged portion 133a, and thus the second arm portion 140 can be easily secured to the enlarged portion 133a.

Subsequently, as illustrated in FIG. 5D, the operator, etc. passes the second arm portion 140 between the thumb f1 and the index finger f2 of the right hand H1 of the patient to dispose the second arm end portion 143 on the side of the dorsal side Hb of the hand H of the patient. Subsequently, the operator, etc. secures the first inclined portion 141a and the second arm end portion 143 via the respective securing members 152 and 156 by bringing the sixth securing member 156 (female side of the surface fastener) disposed on the inner surface of the second arm end portion 143 into contact with the second securing member 152 (male side of the surface fastener) disposed on the outer surface of the first inclined portion 141a on the side of the dorsal side Hb of the right hand H1 of the patient.

Subsequently, the operator, etc. wraps a portion (surplus portion) of the second arm end portion 143 not secured to the first inclined portion 141a along a circumferential direction of the right hand H1 of the patient, thereby further securing the second arm end portion 143 to the enlarged portion 133a. In this way, the hemostatic device 100 can prevent the second arm end portion 143 from being caught in a surrounding article, etc. while the hemostatic device 100 is attached to the operator, etc.

The operator, etc. can secure the hemostatic device 100 to the right hand H1 of the patient by the above procedure. FIG. 5E illustrates a state in which the hemostatic device 100 is attached to the right hand H1 of the patient. FIG. 5E illustrates a state in which the introducer 200 is removed from the puncture site t1.

Next, the operator, etc. connects the syringe to the injection portion 191 and injects air into the first inflatable portion 170 to inflate the first inflatable portion 170 and the second inflatable portion 180. In the hemostatic device 100, when the first inflatable portion 170 and the second inflatable portion 180 are inflated, the first inflatable portion 170 applies a compressive force to the puncture site t1. After inflating the respective inflatable portions 170 and 180, as illustrated in FIG. 5E, the operator, etc. removes the sheath tube of the introducer 200 from the puncture site t1. In this instance, as illustrated in FIG. 5D, since the second arm portion 140 is disposed between the thumb f1 and the index finger f2 of the right hand H1 of the patient, the hemostatic device 100 does not have an arm portion protruding from the main body 120 in a region where the curved portion 125a of the support member 125 and the second inflatable portion 180 are located in the main body 120. For this reason, after inflating the respective inflatable portions 170 and 180, as illustrated in FIG. 5E, the operator, etc. can remove the sheath tube of the introducer 200 from the puncture site t1.

The operator, etc. confirms that there is no bleeding from the puncture site t1 during hemostasis using the hemostatic device 100. When there is bleeding from the puncture site t1, the operator, etc. adjusts the amount of air injected into each of the inflatable portions 170 and 180.

After a certain period of time from the start of hemostasis, the operator, etc. gradually depressurizes each of the inflatable portions 170 and 180 to confirm that hemostasis is properly performed on the puncture site t1. After the hemostasis on the puncture site t1 is completed, the operator, etc. sufficiently depressurizes each of the inflatable portions 170 and 180. Then, the operator, etc. releases securing of the hemostatic device 100 by the first arm portion 130 and the second arm portion 140, and removes the hemostatic device 100 from the hand H of the patient.

As illustrated in FIG. 5E, during hemostasis, the hemostatic device 100 is firmly secured to the right hand H1 of the patient by the second arm portion 140 passed between the thumb f1 and the index finger f2 of the right hand H1 of the patient while being wrapped around the limb of the patient. For this reason, since each finger is not covered by the covering member 110 when the hemostatic device 100 is attached to the right hand H1, the patient can freely move the finger while hemostasis is performed.

By proceeding with an attaching operation according to a procedure described with reference to FIGS. 5A to 5E, the operator, etc. can use the first arm portion 130 and the second arm portion 140 to easily attach the hemostatic device 100 to the patient in a short time. Further, the hemostatic device 100 can secure the covering member 110 to the right hand H1 of the patient using the securing members 151, 152, 153, 154, 155, and 156 disposed on the respective arm portions 130 and 140. Therefore, the hemostatic device 100 can reduce a burden on the skin of the patient during attachment when compared to a hemostatic device secured to the right hand H1 or the forearm A of the patient using a seal member, etc. provided with an adhesive material.

Further, since the hemostatic device 100 includes the first inflatable portion 170 as a member that applies a compressive force to the puncture site t1, the compressive force can be easily adjusted by adjusting the internal pressure of the first inflatable portion 170. Further, in the hemostatic device 100, even when the first inflatable portion 170 is deformed to change the internal pressure following movement of the right hand H1 when the patient moves the right hand H1, the deformable second inflatable portion 180 mitigates the change in the internal pressure of the first inflatable portion 170. Therefore, the first inflatable portion 170 has a high following property (conformability) to movement of the right hand H1 of the patient, and compression on the puncture site t1 by the first inflatable portion 170 can be appropriately maintained.

Further, the hemostatic device 100 is configured to cover only a part of the right hand H1 of the patient by the covering member 110, and is not configured to cover the entire right hand H1. For this reason, when the patient moves the right hand H1 in a state where the hemostatic device 100 is attached to the right hand H1 of the patient, it is possible to prevent movement of the right hand H1 of the patient from being transmitted to the entire hemostatic device 100. Therefore, the hemostatic device 100 can suppress position shift from the right hand H1 of the patient when the patient moves the right hand H1 in the state where the hemostatic device 100 is attached to the right hand H1 of the patient.

The hemostatic device 100 can be attached to the left hand H2 of the patient, for example, without changing the configuration of the hemostatic device 100. Hereinafter, a description will be given of an example of a procedure for attaching the hemostatic device 100 to the left hand H2 of the patient on which the puncture site t1 is formed. A detailed description of content overlapping with the above-described example of the procedure for attaching the hemostatic device 100 to the right hand H1 is not repeated.

As illustrated in FIG. 7A, the operator, etc. disposes the main body 120 of the covering member 110 so as to overlap the side of the dorsal side Hb of the right hand H2.

Subsequently, as illustrated in FIG. 7B, the operator, etc. wraps the inclined portion (the first inclined portion 141a and the second inclined portion 141b) 141 of the second arm portion 140 along the left hand H2 of the patient while wrapping the enlarged portion 133a of the first arm portion 130 around the left hand H2 of the patient. In this instance, the operator, etc. secures the enlarged portion 133a and the second arm portion 140 via the respective securing members 151 and 155 by bringing the fifth securing member 155 (female side of the surface fastener) disposed on the inner surface of the inclined portion 141 into contact with the first securing member 151 (male side of the surface fastener) disposed on the outer surface of the enlarged portion 133a of the first arm portion 130 on the palm Hp side of the left hand H2 of the patient.

Subsequently, as illustrated in FIG. 7C, the operator, etc. disposes the second arm portion 140 on the side of the dorsal side Hb of the left hand H2 of the patient. In this instance, the operator, etc. secures the first arm portion 130 and the second arm portion 140 via the respective securing members 151 and 155 on the side of the dorsal side Hb of the left hand H2 by bringing the fifth securing member 155 (female side of the surface fastener) disposed on the inner surface of the inclined portion 141 into contact with the first securing member 151 (male side of the surface fastener) disposed on the outer surface of the inclined portion 131 of the first arm portion 130 on the side of the dorsal side Hb of the left hand H2 of the patient.

Subsequently, as illustrated in FIG. 7D, the operator, etc. passes the second arm portion 140 between the thumb f1 and the index finger f2 of the left hand H2 of the patient, and disposes the second arm end portion 143 on the palm Hp side of the left hand H2 of the patient. In this instance, the operator, etc. secures the first inclined portion 141a and the second arm end portion 143 via the respective securing members 152 and 156 by bringing the sixth securing member 156 (female side of the surface fastener) disposed on the inner surface of the second arm end portion 143 into contact with the second securing member 152 (male side of the surface fastener) disposed on the outer surface of the inclined portion 141 on the palm Hp side of the left hand H2 of the patient.

The operator, etc. can secure the hemostatic device 100 to the left hand H2 of the patient by the above procedure.

Hereinafter, effects of the present embodiment will be described.

The hemostatic device 100 according to the present embodiment includes the covering member 110 disposed to cover the puncture site t1 on the right hand H1 (or left hand H2) of the patient, the plurality of securing members 151, 152, 153, 154, 155, and 156 configured to secure the covering member 110 in a state where the covering member 110 covers the puncture site t1, and the inflatable member 160 connected to the covering member 110 and configured to be inflated by injection of a fluid. The covering member 110 has the main body 120 to which the inflatable member 160 is connected, the first arm portion 130 protruding from the main body 120, and the second arm portion 140 protruding from the main body 120 while forming an obtuse angle with the longitudinal direction of the first arm portion 130. Further, the first arm portion 130 has the enlarged portion 133a protruding in the width direction of the first arm portion 130 at the end portion 133 of the first arm portion 130. Furthermore, the second arm portion 140 is longer than the first arm portion 130 and is configured to be secured to the enlarged portion 133a and secured to the second arm portion 140 by passing between the fingers f1 and f2 of the patient in a state where the second arm portion 140 is wrapped around the limb of the patient.

In the hemostatic device 100 configured as described above, it is possible to secure the main body 120 in which the inflatable member 160 is disposed to the limb by disposing a part of the second arm portion 140 between the adjacent fingers f1 and f2 of the patient and connecting the enlarged portion 133a and the second arm portion 140 while wrapping the first arm portion 130 and the second arm portion 140 around the limb of the patient. The first arm portion 130 and the second arm portion 140 are fastened to the limb by connecting the enlarged portion 133a and the second arm portion 140, and the main body 120 is secured to the limb. Further, by disposing the second arm portion 140 between the adjacent fingers f1 and f2 of the patient, it is possible to suppress position shift of the inflatable member 160 disposed in the main body 120 with respect to the puncture site t1. Further, the hemostatic device 100 has a simple securing structure capable of tightening the inflatable member 160 to the hand H1 of the patient by connecting the two arm portions 130 and 140 protruding from the main body 120. For this reason, the hemostatic device 100 can reduce the number of arm portions installed for securing the inflatable member 160. Therefore, the hemostatic device 100 can suppress an increase in arm portions that may interfere with a medical device such as the introducer 200 when the hemostatic device 100 is attached to the hand H1 of the patient, and it is possible to easily remove the medical device from the puncture site t1 even after the hemostatic device 100 is attached. Further, a movable range of the right hand H1 (or the left hand H2) increases from the wrist side to the fingertip side of the right hand H1. In the hemostatic device 100, the first arm portion 130 and the second arm portion 140 extend from the fingertip side to the wrist side of the right hand H1 while forming an obtuse angle, so that the first arm portion 130 and the second arm portion 140 can be connected on the wrist side of the right hand H1 of the patient. Therefore, in the hemostatic device 100, the first arm portion 130 and the second arm portion 140 can be secured on the wrist side where the movable range is small, and thus the main body 120 can be secured to the limb while maintaining the movable range on the fingertip side of the right hand H1. An outer circumference of the right hand H1 becomes larger from the wrist side to the fingertip side of the right hand H1 in a state where the right hand H1 is spread. Therefore, since the first arm portion 130 and the second arm portion 140 extend in a direction opposite to a direction in which the outer circumference of the right hand H1 becomes larger, the first arm portion 130 and the second arm portion 140 can be reliably connected on the wrist side of the right hand H1 in the state where the hemostatic device 100 is attached. Furthermore, in the hemostatic device 100, even in the case where the patient moves the hand H with the inflatable member 160 inflated, when the first arm portion 130 and the second arm portion 140 are secured in a state of being tightened to the limb of the patient, it is possible to prevent rising of the distal side (fingertip side) of the main body 120 to which the inflatable member 160 is connected, and to appropriately maintain the compressive force of the inflatable member 160 on the puncture site t1 formed on the dorsal side Hb of the hand H1 of the patient.

Further, the main body 120 has first regions 121a and 121b and the second region 122 in which the inflatable member 160 is disposed at a location different from the first regions 121a and 121b. The first region 121a is disposed between the second region 122 and the first arm portion 130, and the first region 121b is disposed between the second region 122 and the second arm portion 140. Further, the second region 122 includes the support member 125 having a higher rigidity than that of the first regions 121a and 121b. Therefore, the hemostatic device 100 can prevent the inflatable member 160 from rising or lifting from the dorsal side Hb of the hand H of the patient by the support member 125 while hemostasis is performed. As a result, the hemostatic device 100 can suitably apply a compressive force from the inflatable member 160 to the puncture site t1 while being attached to the right hand H1 of the patient.

Further, the support member 125 has the curved portion 125a formed on the distal side of the main body 120. For this reason, in the support member 125, when the inflatable member 160 is inflated, a direction in which the inflatable member 160 applies a compressive force to the right hand H1 of the patient is directed in an oblique direction toward the puncture site t1. For this reason, the hemostatic device 100 can more effectively apply a compressive force to the puncture site t1. Further, the curved portion 125a of the support member 125 directs the compressive force of the inflatable member 160 toward the center portion of the support member 125 by wrapping the end portion of the inflatable member 160 on the distal side when the inflatable member 160 inflates, and thus the compressive force of the inflatable member 160 is inhibited from escaping to the outside of the support member 125. For this reason, the hemostatic device 100 can suppress a decrease in the compressive force to the puncture site t1 by the inflatable member 160, and thus can appropriately maintain the compressive force of the inflatable member 160 to the puncture site t1.

Further, the inflatable member 160 has the first inflatable portion 170 and the deformable auxiliary member 180 having a smaller outer shape than that of the first inflatable portion 170, and the auxiliary member 180 overlaps the first inflatable portion 170 on the distal side of the main body 120. In the hemostatic device 100 configured as described above, when the first inflatable portion 170 inflates, the second inflatable portion 180 inhibits the first inflatable portion 170 from rising or lifting away from the body surface of the patient. Therefore, when the first inflatable portion 170 inflates, the first inflatable portion 170 can effectively apply the compressive force to the puncture site t1. Further, when the patient bends a finger (for example, the thumb f1) or the wrist downward (to the palm Hp side of the hand H), the hemostatic device 100 maintains a state in which the auxiliary member 180 is deformed by following movement of the finger or the wrist and the auxiliary member 180 applies the compressive force to the puncture site t1.

Further, the auxiliary member is the second inflatable portion 180 configured to be inflated by injection of a fluid, and the lumen 181 of the second inflatable portion 180 communicates with the lumen 171 of the first inflatable portion 170. Since the auxiliary member 180 includes the second inflatable portion 180 which is inflatable, it is possible to improve the following property (conformability) of the second inflatable portion 180 to movement of the right hand H1. Further, since the lumen 171 of the first inflatable portion 170 and the lumen 181 of the second inflatable portion 180 communicate with each other, the first inflatable portion 170 and the second inflatable portion 180 can be easily inflated.

In addition, the second inflatable portion 180 is disposed to have bilateral symmetry with respect to the center line d3 of the first inflatable portion 170, and the securing members 151 and 152 for securing the second arm portion 140 are disposed on the outer surface of the first arm portion 130 and the outer surface of the second arm portion 140 opposing each other with the main body 120 interposed therebetween. For this reason, when the hemostatic device 100 is attached to the right hand H1 of the patient, the operator, etc. can secure the second arm portion 140 to the enlarged portion 133a through the respective securing members 151 and 152 while disposing the first inflatable portion 170 on the puncture site t1 so that the puncture site t1 is located on the center line d3 of the auxiliary member 180. Similarly, when the hemostatic device 100 is attached to the left hand H2 of the patient, the operator, etc. can secure the second arm portion 140 to the enlarged portion 133a through the respective securing members 151 and 152 while disposing the first inflatable portion 170 on the puncture site t1 so that the puncture site t1 is located on the center line d3 of the auxiliary member 180. Therefore, the operator, etc. can attach the hemostatic device 100 to both the right hand H1 and the left hand H2 of the patient.

Further, the enlarged portion 133a protrudes or extends toward the distal side of the main body 120 in a direction intersecting the extending direction of the first arm portion 130. Therefore, when the second arm portion 140 is secured to the enlarged portion 133a, the operator, etc. can confirm the position of the enlarged portion 133a disposed on the palm Hp side of the hand H and the shape of the enlarged portion 133a by the hand feel, etc., and thus the second arm portion 140 can be easily secured to the enlarged portion 133a.

Further, the second arm portion 140 has the inclined portion 141 and the second arm end portion 143 which is continuously formed with the inclined portion 141 and forms an end portion of the second arm portion 140, and the width of the inclined portion 141 is larger than the width of the second arm end portion 143. Therefore, when the second arm portion 140 is secured to the enlarged portion 133a, by wrapping the inclined portion 141 around the hand H of the patient, the operator, etc. can more reliably dispose the inclined portion 141 with respect to the enlarged portion 133a, and easily secure the second arm portion 140.

Further, the hemostatic device 100 has the injection portion 191 for injecting a fluid into the inflatable member 160. The cushioning member 192 having an inflatable space is disposed between the injection portion 191 and the inflatable member 160. In the hemostatic device 100, when the patient moves the right hand H1 while the hemostatic device 100 is attached to the patient, the inflatable member 160 (the first inflatable portion 170 and the second inflatable portion 180) that compresses the puncture site t1 on the right hand H1 is deformed. When the inflatable member 160 is deformed, if there is no escape place for air in the lumen of the inflatable member 160 (the lumen 171 of the first inflatable portion 170 and the lumen 181 of the second inflatable portion 180), deformation of the inflatable member 160 is hindered. For this reason, the patient has a limited movable range for the right hand H1. The cushioning member 192 included in the hemostatic device 100 allows air to move from the lumen of the inflatable member 160 to the cushioning member 192 when the patient moves the right hand H1. For this reason, the patient can prevent the movable range from being restricted by the inflatable member 160 when the right hand H1 is moved. When the patient returns the right hand H1 from the deformed state to the original state, air moves from the cushioning member 192 to the inflatable member 160, and thus the compressive force can be effectively applied to the puncture site t1 from the inflatable member 160.

(Modification)

Next, a description will be set forth of a modification of the hemostatic device according to the first embodiment described above. In the description of the modification, features that are the same or similar to those described above are identified by the same reference numerals and a detailed description of such features is not repeated. Thus, content not particularly described in the description of the modification can be regarded as the same as that in the first embodiment.

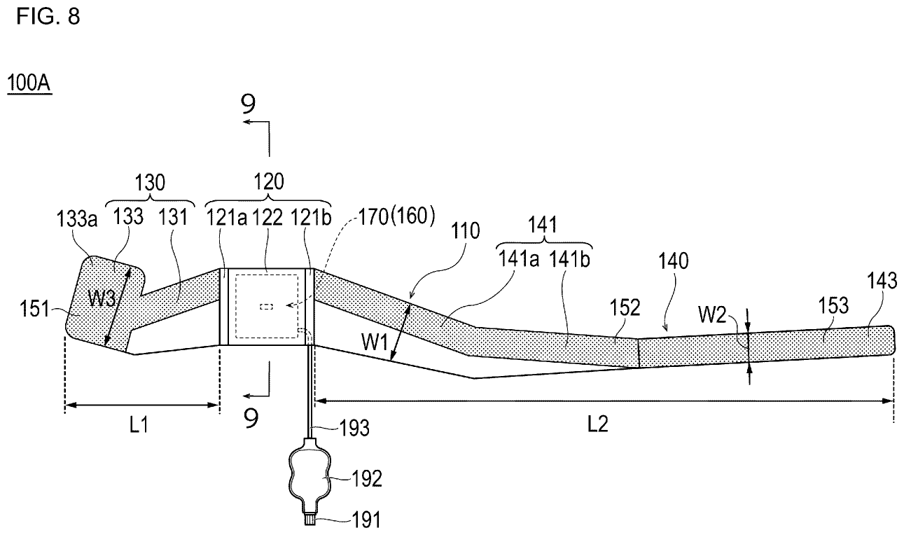

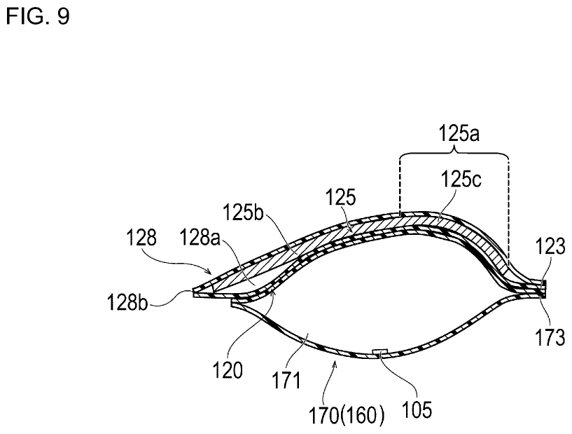

FIG. 8 is a plan view of a hemostatic device 100A seen from the outer surface side of the main body 120 of the covering member 110, and FIG. 9 is a cross-sectional view of the hemostatic device 10A taken along the section line 9-9 in FIG. 8.

In the hemostatic device 100A according to the modification, the inflatable member 160 includes only the first inflatable portion 170. That is, the inflatable member 160 does not include the second inflatable portion 180. Further, in the hemostatic device 100A, as illustrated in FIG. 9, the end portion 173 of the first inflatable portion 170 on the distal side included in the inflatable member 160 is directly fixed to the inner surface of the covering member 110. Other configurations, features, or aspects of the hemostatic device 100A are substantially the same as those of the hemostatic device 100 described above.

In the hemostatic device 100A according to the modification, similarly to the hemostatic device 100 described above, position shift of the inflatable member 160 disposed on the main body 120 with respect to the puncture site t1 can be suppressed by connecting the enlarged portion 133a wrapped around the limb of the patient and the second arm portion 140, and securing a part of the second arm portion 140 passed between the fingers f1 and f2 of the patient to the second arm portion 140. Further, since the hemostatic device 100A has a simple securing structure capable of tightening the inflatable member 160 to the right hand H1 of the patient by connecting the two arm portions 130 and 140 protruding from the main body 120, the hemostatic device 100A can reduce the number of arm portions installed for securing the inflatable member 160. Therefore, the hemostatic device 100A can suppress an increase in arm portions that may interfere with a medical device such as the introducer 200 when the hemostatic device 100A is attached to the hand H1 of the patient, and it is possible to easily remove the medical device from the puncture site t1 even after the hemostatic device 100A is attached. Further, a movable range of the right hand H1 (or the left hand H2) of the patient increases from the wrist side to the fingertip side of the right hand H1. In the hemostatic device 100A, the first arm portion 130 and the second arm portion 140 extend from the fingertip side to the wrist side of the right hand H1 while forming an obtuse angle, so that the first arm portion 130 and the second arm portion 140 can be connected on the wrist side of the right hand H1 of the patient. Therefore, in the hemostatic device 100A, the first arm portion 130 and the second arm portion 140 can be secured on the wrist side where the movable range is small, and thus the main body 120 can be secured to the limb while maintaining the movable range on the fingertip side of the right hand H1. The outer circumference of the right hand H1 becomes larger from the wrist side to the fingertip side of the right hand H1 in a state where the right hand H1 is spread. Therefore, since the first arm portion 130 and the second arm portion 140 extend in a direction opposite to a direction in which the outer circumference of the right hand H1 becomes larger, the first arm portion 130 and the second arm portion 140 can be reliably connected on the wrist side of the right hand H1 in the state where the hemostatic device 100A is attached. Furthermore, in the hemostatic device 100A, even in the case where the patient moves the right hand H1 with the inflatable member 160 inflated, when the first arm portion 130 and the second arm portion 140 are secured in a state of being tightened to the limb of the patient, it is possible to prevent rising of the distal side (fingertip side) of the main body 120 to which the inflatable member 160 is connected, and to appropriately maintain the compressive force of the inflatable member 160 on the puncture site t1 formed on the dorsal side Hb of the right hand H1 of the patient.

### Second Embodiment

Next, a description will be given of a hemostatic device according to a second embodiment. In description of the second embodiment, features that are the same or similar to those described above are identified by the same reference numerals and a detailed description of such features is not repeated. In addition, content not particularly described in the description of the second embodiment can be regarded as the same as that in the first embodiment.

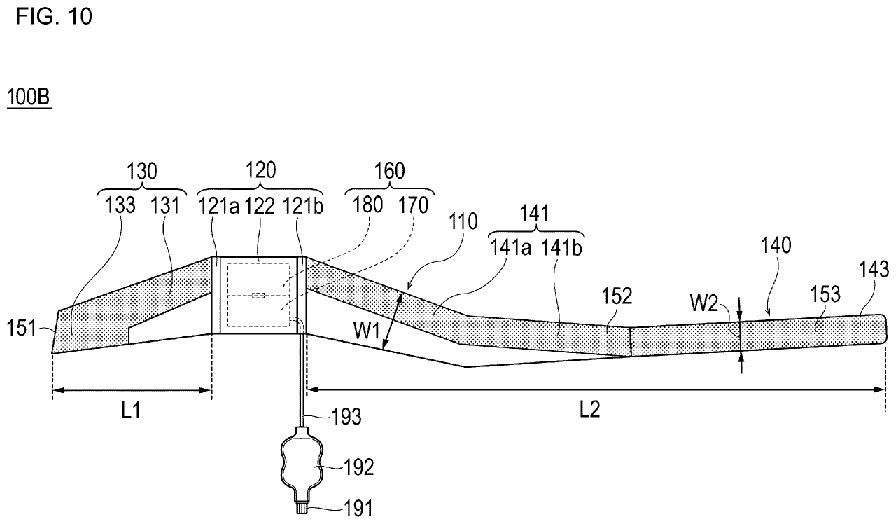

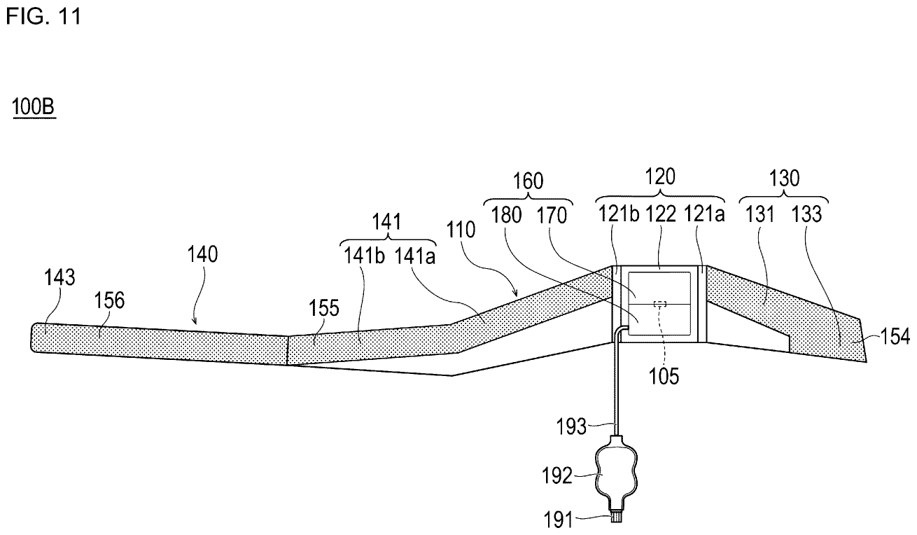

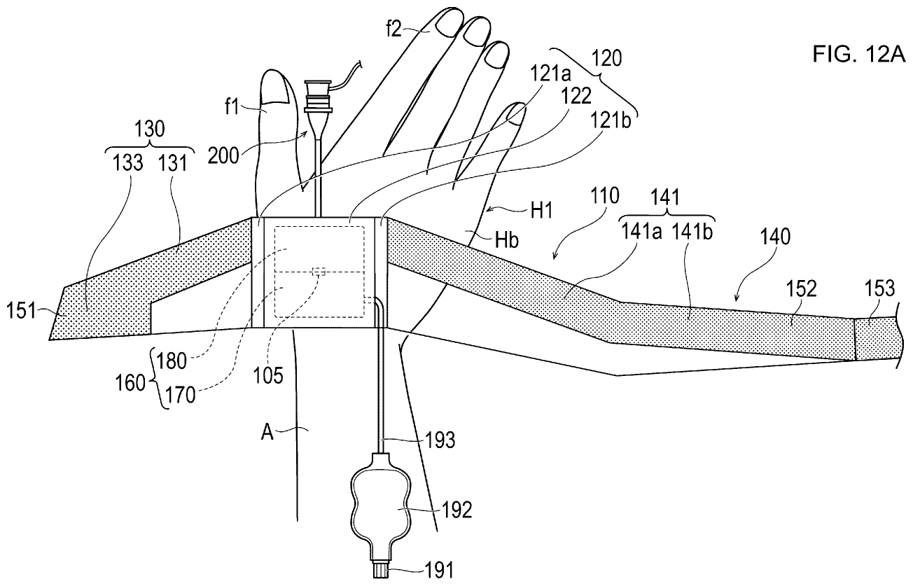

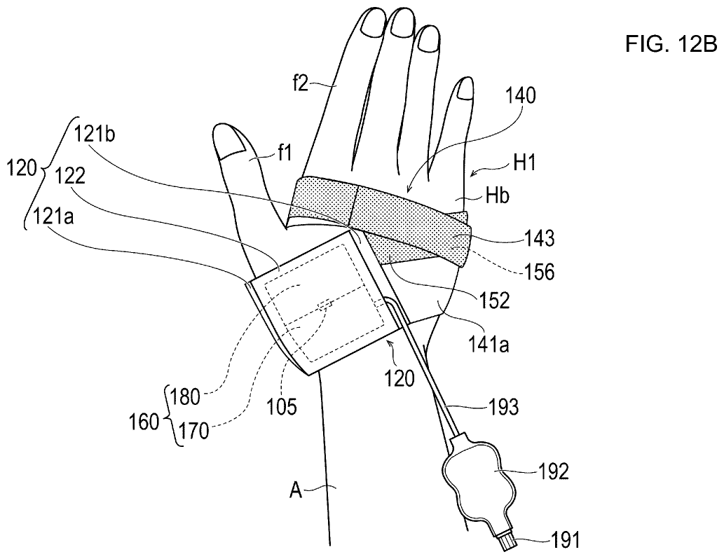

FIG. 10 is a plan view of a hemostatic device 100B seen from the outer surface side of the main body 120 of the covering member 110, and FIG. 11 is a plan view of the hemostatic device 100B seen from the inner surface side of the main body 120 of the covering member 110. FIGS. 12A and 12B are diagrams briefly illustrating a usage example of the hemostatic device 100B.

As illustrated in FIGS. 10 and 11, in the hemostatic device 100B according to the second embodiment, the first arm portion 130 does not include the enlarged portion 133a (see FIGS. 1 and 2). In this respect, the second embodiment differs from the hemostatic device 100 according to the first embodiment.

The end portion 133 of the first arm portion 130 of the hemostatic device 100B is continuously connected to the inclined portion 131, and extends linearly along a direction substantially the same as the extending direction of the inclined portion 131.

As illustrated in FIG. 10, a first securing member 151 is disposed on a part of the outer surface of the first arm portion 130 on the distal side. Further, as illustrated in FIG. 11, a fourth securing member 154 is disposed on a part of the inner surface of the first arm portion 130 on the distal side.

FIG. 12A illustrates a state before the hemostatic device 100B is attached to the limb of the patient, and FIG. 12B illustrates a state in which the hemostatic device 1006 is attached to the limb of the patient. The hemostatic device 100B can be attached to the right hand H1 or the left hand H2 of the patient on which the puncture site t1 is formed by substantially the same procedure as the attachment procedure of the hemostatic device 100 according to the first embodiment described above. The procedure for attaching the hemostatic device 1006 to the right hand H1 of the patient will be outlined below.

As illustrated in FIG. 12A, the operator, etc. disposes the main body 120 of the covering member 110 so that the main body 120 overlaps the side of the dorsal side Hb of the right hand H1. Subsequently, the operator, etc. wraps the inclined portion (the first inclined portion 141a and the second inclined portion 141b) 141 of the second arm portion 140 along or around the right hand H1 of the patient while wrapping the end portion 133 of the first arm portion 130 around the right hand H1 of the patient. In this instance, the operator, etc. secures the end portion 133 and the second arm portion 140 via the respective securing members 151 and 155 by bringing the fifth securing member 155 (female side of the surface fastener) disposed on the inner surface of the inclined portion 141 into contact with the first securing member 151 (male side of the surface fastener) disposed on the outer surface of the end portion 133 of the first arm portion 130 on the palm Hp side of the right hand H1 of the patient.

Subsequently, the operator, etc. passes the second arm portion 140 between the thumb f1 and the index finger f2 of the right hand H1 of the patient, and disposes the second arm end portion 143 on the side of the dorsal side Hb of the right hand H1 of the patient. In this instance, the operator, etc. secures the first inclined portion 141a and the second arm end portion 143 via the respective securing members 152 and 156 by bringing the sixth securing member 156 (female side of the surface fastener) disposed on the inner surface of the second arm end portion 143 into contact with the second securing member 152 (male side of the surface fastener) disposed on the outer surface of the inclined portion 141 on the side of the dorsal side Hb of the right hand H1 of the patient.

By the above procedure, as illustrated in FIG. 12B, the operator, etc. can secure the hemostatic device 100 to the right hand H1 of the patient.

As described above, the hemostatic device 100B according to the second embodiment includes the covering member 110 disposed to cover the puncture site t1 on the right hand H1 (or left hand H2) of the patient, the plurality of securing members 151, 152, 153, 154, 155, and 156 that is configured to secure the covering member 110 in a state where the covering member 110 covers the puncture site t1, and the inflatable member 160 connected to the covering member 110 and configured to be inflated by injection of a fluid. Further, the covering member 110 has the main body 120 to which the inflatable member 160 is connected, the first arm portion 130 protruding from the main body 120, and the second arm portion 140 protruding from the main body 120 while forming an obtuse angle with the longitudinal direction of the first arm portion 130. Further, the inflatable member 160 has the first inflatable portion 170 and the deformable auxiliary member 180 which has a smaller outer shape than that of the first inflatable portion 170 and is biased or offset toward the distal side (one end side) of the first inflatable portion 170. In the covering member 110, the first arm portion 130 and the second arm portion 140 can be connected by the securing members 151 and 155 in a state where the covering member 110 is wrapped around the limb of the patient, and the auxiliary member 180 is disposed between the first arm portion 130 and the second arm portion 140.

In the hemostatic device 100B configured as described above, since the first arm portion 130 and the second arm portion 140 protrude from the main body 120 so as to form an obtuse angle, when the hemostatic device 1006 is attached to the right hand H1 of the patient, the arm portions 130 and 140 can be connected so that the entire finger of the patient is not covered by the covering member 110. For this reason, the patient is less likely to be restricted in movement of the right hand H1 even when the hemostatic device 1006 is attached, and thus a degree of freedom on the fingertip side can be increased. Further, since the hemostatic device 1006 has the deformable auxiliary member 180 located on the distal side of the first inflatable portion 170, when the first inflatable portion 170 is inflated, the auxiliary member 180 presses the first inflatable portion 170 against the limb of the patient to inhibit the first inflatable portion 170 from rising in a direction away from the puncture site t1 on the right hand H1 of the patient. As a result, the hemostatic device 1006 can maintain an appropriate compressive force on the puncture site t1 while increasing the degree of freedom on the fingertip side. Furthermore, in the hemostatic device 1006, since the first arm portion 130 and the second arm portion 140 extend from the fingertip side to the wrist side of the right hand H1 while forming an obtuse angle, the first arm portion 130 and the second arm portion 140 can be secured on the wrist side where the movable range is small. Therefore, the main body 120 can be appropriately secured to the limb while maintaining the movable range on the fingertip side of the right hand H1. Further, in the hemostatic device 1006, since the first inflatable portion 170 can be secured to the puncture site t1 by the two arm portions including the first arm portion 130 and the second arm portion 140 and the auxiliary member 180, it is unnecessary to add different arm portions between the first arm portion 130 and the second arm portion 140. Therefore, the operator, etc. can easily remove the medical device such as the introducer 200 after attaching the hemostatic device 1006. Further, since the first arm portion 130 and the second arm portion 140 are connected in a state of being wrapped around the right hand H1 of the patient, the first arm portion 130 and the second arm portion 140 are pressed against the right hand H1 of the patient while pulling the main body 120 located therebetween toward the both side portion sides (vertical direction in FIG. 10). Since the auxiliary member 180 is located between the first arm portion 130 and the second arm portion 140, the inflatable member 160 can be reliably pressed against the body surface of the right hand H1 by connection of the first arm portion 130 and the second arm portion 140. Therefore, the hemostatic device 1006 can press the first inflatable portion 170 against the limb of the patient by the auxiliary member 180, and reliably inhibit the first inflatable portion 170 from rising in the direction away from the puncture site t1 on the right hand H1 of the patient. In this way, the hemostatic device 100B can reliably press the first inflatable portion 170 against the right hand H1 of the patient and maintain an appropriate compressive force on the puncture site t1 while increasing the degree of freedom on the fingertip side during attachment of the hemostatic device 1006.

Further, the main body 120 has the first regions 121a and 121b and the second region 122 in which the inflatable member 160 is disposed unlike the first regions 121a and 121b. The first region 121a is disposed between the second region 122 and the first arm portion 130, and the first region 121b is disposed between the second region 122 and the second arm portion 140. Further, the second region 122 includes the support member 125 having a higher rigidity than that of the first regions 121a and 121b. Therefore, the hemostatic device 1006 can prevent the inflatable member 160 from rising from the dorsal side Hb of the hand H of the patient by the support member 125 while hemostasis is performed. As a result, the hemostatic device 100 can suitably apply a compressive force from the inflatable member 160 to the puncture site t1 while being attached to the hand H of the patient.

Further, the support member 125 has the curved portion 125a formed on the distal side of the main body 120. For this reason, in the support member 125, when the inflatable member 160 is inflated, a direction in which the inflatable member 160 applies a compressive force to the right hand H1 of the patient is directed in an oblique direction toward the puncture site t1. For this reason, the hemostatic device 100 can more effectively apply a compressive force to the puncture site t1. Further, when the curved portion 125a of the support member 125 wraps or encircles the end portion of the inflatable member 160 on the distal side, the inflatable member 160 directs the compressive force toward the central portion of the support member 125, and thus the compressive force of the second inflatable portion 180 is inhibited from escaping to the outside of the support member 125. In this way, even in the case where a gap is generated between the first arm portion 130 and the right hand H1 of the patient, when the curved portion 125a presses the second inflatable portion 180 against the skin of the right hand H1 of the patient, it is possible to prevent the first inflatable portion 170 from rising or lifting away from the skin, and it is possible to prevent a decrease in the compressive force applied to the puncture site t1 by the inflatable member 160.

Further, the auxiliary member is the second inflatable portion 180 configured to be inflated by injection of a fluid, and the lumen 181 of the second inflatable portion 180 communicates with the lumen 171 of the first inflatable portion 170. Since the auxiliary member 180 includes the second inflatable portion 180 which is inflatable, it is possible to improve the following property of the second inflatable portion 180 to movement of the right hand H1. Further, since the lumen 171 of the first inflatable portion 170 and the lumen 181 of the second inflatable portion 180 communicate with each other, the first inflatable portion 170 and the second inflatable portion 180 can be easily inflated.

Further, the second arm portion 140 is longer than the first arm portion 130, and is configured to be secured to the second arm portion 140 by passing between the fingers f1 and f2 of the patient in a state where the second arm portion 140 is wrapped around the limb of the patient. For this reason, the hemostatic device 100B can secure the main body 120 in which the inflatable member 160 is disposed to the limb by disposing a part of the second arm portion 140 between the adjacent fingers f1 and f2 of the patient and connecting parts of the second arm portion 140 to each other while wrapping the first arm portion 130 and the second arm portion 140 around the limb of the patient. The first arm portion 130 and the second arm portion 140 are fastened to the limb by connecting the parts of the second arm portions 140 to each other, and the main body 120 is secured to the limb. By disposing the second arm portion 140 between the adjacent fingers f1 and f2 of the patient, it is possible to effectively suppress position shift of the inflatable member 160 disposed on the main body 120 with respect to the puncture site t1.

In addition, the second inflatable portion 180 is disposed to have bilateral symmetry with respect to the center line d3 of the first inflatable portion 170, and the securing members 151 and 152 for securing the second arm portion 140 are disposed on the outer surfaces of the first arm portion 130 and the second arm portion 140 opposing to each other with the main body 120 interposed therebetween. For this reason, when the hemostatic device 100 is attached to the right hand H1 of the patient, the operator, etc. can secure the respective arm portions 130 and 140 through the respective securing members 151 and 152 while disposing the first inflatable portion 170 on the puncture site t1 so that the puncture site t1 is located on the center line d3 of the auxiliary member 180. Similarly, when the hemostatic device 100 is attached to the left hand H2 of the patient, the operator, etc. can secure the respective arm portions 130 and 140 through the respective securing members 151 and 152 while disposing the first inflatable portion 170 on the puncture site t1 so that the puncture site t1 is located on the center line d3 of the auxiliary member 180. Therefore, the operator, etc. can attach the hemostatic device 100 to both the right hand H1 and the left hand H2 of the patient.

Further, the second arm portion 140 has the inclined portion 141 and the second arm end portion 143 which is continuously formed with the inclined portion 141 and forms an end portion of the second arm portion 140, and the width of the inclined portion 141 is larger than the width of the second arm end portion 143. Therefore, when the inclined portion 141 is wrapped around the hand H of the patient, the operator, etc. can more reliably dispose the inclined portion 141 on the second arm end portion 143, and easily secure the second arm portion 140.

Further, the hemostatic device 100B has the injection portion 191 for injecting a fluid into the inflatable member 160. The cushioning member 192 having an inflatable space is disposed between the injection portion 191 and the inflatable member 160. In the hemostatic device 1006, when the patient moves the right hand H1 while the hemostatic device 100B is attached to the patient, the inflatable member 160 (the first inflatable portion 170 and the second inflatable portion 180) that compresses the puncture site t1 on the right hand H1 is deformed. When the inflatable member 160 is deformed, if there is no escape place for air in the lumen of the inflatable member 160 (the lumen 171 of the first inflatable portion 170 and the lumen 181 of the second inflatable portion 180), deformation of the inflatable member 160 is hindered. For this reason, the patient has a limited movable range for the right hand H1. The cushioning member 192 included in the hemostatic device 1006 allows air to move from the lumen of the inflatable member 160 to the cushioning member 192 when the patient moves the right hand H1. For this reason, the patient can prevent the movable range from being restricted by the inflatable member 160 when the right hand H1 is moved. When the patient returns the right hand H1 from the deformed state to the original state, air moves from the cushioning member 192 to the inflatable member 160, and thus the compressive force can be effectively applied to the puncture site t1 from the inflatable member 160.

Even though the hemostatic device and usage method disclosed here have been described above through several embodiments representing examples of the inventive hemostatic device and method disclosed here, the invention is not limited to the specific content described in this specification, and can be appropriately modified within the claim scope.

The auxiliary member is not limited to the inflatable member described in each embodiment. For example, the auxiliary member may include a member made of a resin material such as plastic, gel, etc., a member containing gel whose moisture content decreases over time to gradually reduce a compressive force, an elastic material such as a sponge-like substance, an aggregate of fibers such as cotton, metal, a member having a predetermined three-dimensional shape (sphere, ellipsoid, triangular pyramid, etc.), an appropriate combination thereof, etc.

In addition, the shape and dimensions of each portion of the hemostatic device are not particularly limited as long as the inflatable member can be disposed at the puncture site while wrapping the first arm portion and the second arm portion around the limb, and changes can be made as appropriate.

The detailed description above describes embodiments of a hemostatic device and method of performing hemostasis representing examples of the inventive hemostatic device and method disclosed here. The invention is not limited, however, to the precise embodiments, modifications and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A hemostatic device comprising:
a covering member configured to cover a site on a hand of a patient where bleeding is to be stopped;
a securing member connected to the covering member and configured to secure the covering member on the hand of the patient while the covering member is covering the site on the patient's hand where bleeding is to be stopped;
an inflatable member connected to the covering member and configured to be inflated by injection of a fluid;
the covering member including a main body to which the inflatable member is connected, a first arm portion extending away from the main body in a longitudinal direction of extent, and a second arm portion extending away from the main body in a direction forming an obtuse angle with the longitudinal direction of extent of the first arm portion;
the first arm portion including one end portion closest to the main body and an opposite end portion remote from the main body, the first arm portion including an enlarged portion that protrudes in a width direction of the first arm portion at the opposite end portion of the first arm portion; and
the second arm portion being longer than the first arm portion, the second arm portion including a first part configured to be secured to the enlarged portion by way of a first part of the securing member, the second arm portion being configured to pass between fingers of the hand of the patient while the second arm portion is wrapped around a limb of the patient and including a second part configured to be secured to a third part of the second arm portion by way of a second part of the securing member.

2. The hemostatic device according to claim 1,
wherein the main body includes a first region and a second region, the second region being at a location different from the first region, the inflatable member being disposed in the second region,
the first region being disposed between the second region and the first arm portion and between the second region and the second arm portion, and
the second region including a support member that is more rigid than the first region.

3. The hemostatic device according to claim 2, wherein the main body includes a distal side located between the first arm portion and the second arm portion and facing towards tips of the fingers of the hand of the patient when the hemostatic device is mounted on the hand of the patient, the support member possessing a longitudinal extent extending between one end portion closer to the distal side of the main body and an opposite end portion remote from the distal side of the main body, the one end portion of the support member including a curved portion, the curved portion including a concave surface facing towards the inflatable member and a concave surface facing away from the inflatable member.

4. The hemostatic device according to claim 1,
wherein the inflatable member includes a first inflatable portion and a deformable auxiliary member having a smaller outer shape than an outer shape of the first inflatable portion,
the main body including a distal side located between the first arm portion and the second arm portion,
the first inflatable member including one end portion located closer to the distal side of the main body and an opposite end portion, and
the auxiliary member overlapping the one portion of the first inflatable portion.

5. The hemostatic device according to claim 4,
wherein the auxiliary member is a second inflatable portion configured to be inflated by injection of a fluid, the first and second inflatable portions each including a respective lumen that receives the fluid during inflation of the first and second inflatable portions, and
the lumen of the second inflatable portion is in communication with the lumen of the first inflatable portion.

6. The hemostatic device according to claim 4,
wherein the auxiliary member is symmetrical with respect to a center line of the first inflatable portion that passes through a center of the auxiliary member and intersects the distal side of the main body, the auxiliary member also being symmetrical with respect to another line perpendicular to the center line and passing through the center of the auxiliary member, and
the securing member for securing the second arm portion being disposed on outer surfaces of the first arm portion and the second arm portion opposing each other with the main body interposed therebetween.

7. The hemostatic device according to claim 1, wherein the main body includes a distal side located between the first arm portion and the second arm portion and facing towards tips of the fingers of the hand of the patient when the hemostatic device is mounted on the hand of the patient, the distal side of the main body facing in a distal direction, the enlarged portion protruding in a direction intersecting the longitudinal direction of extent of the first arm portion and protruding toward the distal direction.

8. The hemostatic device according to claim 1,
wherein the second arm portion includes an inclined portion and a second arm end portion which is a continuation of the inclined portion and which is an end portion of the second arm portion, and
a width of the inclined portion is larger than a width of the second arm end portion.

9. The hemostatic device according to claim 1,
further comprising an injection portion for injecting the fluid into the inflatable member to inflate the inflatable member, and
a cushioning member that includes an inflatable space, the cushioning member being disposed between the injection portion and the inflatable member.

10. A hemostatic device comprising:
a covering member configured to cover a site on a hand of a patient where bleeding is to be stopped;
a securing member connected to the covering member and configured to secure the covering member on the hand of the patient while the covering member is covering the site on the patient's hand where bleeding is to be stopped;
an inflatable member connected to the covering member and configured to be inflated by injection of a fluid;
the covering member including a main body to which the inflatable member is connected, a first arm portion extending away from the main body in a longitudinal direction of extent, and a second arm portion extending away from the main body in a direction forming an obtuse angle with the longitudinal direction of extent of the first arm portion;
the inflatable member including a first inflatable portion and a deformable auxiliary member which has a smaller outer shape than an outer shape of the first inflatable portion and is offset toward one end side of the first inflatable portion,
the first arm portion and the second arm portion of the covering member are configured to be connectable to one another by the securing member in a state in which the covering member is wrapped around a limb of the patient; and
the auxiliary member being disposed between the first arm portion and the second arm portion.

11. The hemostatic device according to claim 10,
wherein the main body includes a first region and a second region, the second region being at a location different from the first region, the inflatable member being disposed in the second region,
the first region being disposed between the second region and the first arm portion and between the second region and the second arm portion, and
the second region including a support member that is more rigid than the first region.

12. The hemostatic device according to claim 11, wherein the main body includes a distal side located between the first arm portion and the second arm portion and facing towards tips of the fingers of the hand of the patient when the hemostatic device is mounted on the hand of the patient, the support member possessing a longitudinal extent extending between one end portion closer to the distal side of the main body and an opposite end portion remote from the distal side of the main body, the one end portion of the support member including a curved portion, the curved portion including a concave surface facing towards the inflatable member and a concave surface facing away from the inflatable member.

13. The hemostatic device according to claim 10,
wherein the auxiliary member is a second inflatable portion configured to be inflated by injection of a fluid, the first and second inflatable portions each including a respective lumen that receives the fluid during inflation of the first and second inflatable portions, and
the lumen of the second inflatable portion is in communication with the lumen of the first inflatable portion.

14. The hemostatic device according to claim 10, wherein the second arm portion is longer than the first arm portion and is configured to pass between fingers of the patient in a state where the second arm portion is wrapped around the limb of the patient and including a part configured to be secured to an other part of the second arm portion by way of a portion of the securing member.

15. The hemostatic device according to claim 10,
wherein the auxiliary member is symmetrical with respect to a center line of the first inflatable portion that passes through a center of the auxiliary member and intersects a distal side of the main body, the auxiliary member also being symmetrical with respect to another line perpendicular to the center line and passing through the center of the auxiliary member, and
the securing member for securing the second arm portion being disposed on outer surfaces of the first arm portion and the second arm portion opposing to each other with the main body interposed therebetween.

16. The hemostatic device according to claim 10,
wherein the second arm portion includes an inclined portion and a second arm end portion which is a continuation of the inclined portion and which is an end portion of the second arm portion, and
a width of the inclined portion is larger than a width of the second arm end portion.

17. The hemostatic device according to claim 10,
further comprising an injection portion for injecting the fluid into the inflatable member to inflate the inflatable member, and
a cushioning member that includes an inflatable space, the cushioning member being disposed between the injection portion and the inflatable member.

18. A method of producing hemostasis at a puncture site on a hand of a patient where bleeding is to be stopped, the method comprising:
positioning a main body of a covering member in overlying relation to the puncture site on the hand of the patient where bleeding is to be stopped so that an inflatable member underlying the main body portion is positioned relative to the puncture site, the covering member also including a first arm portion extending away from the main body in a longitudinal direction of extent, and a second arm portion extending away from the main body in a direction forming an obtuse angle with the longitudinal direction of extent of the first arm portion, the first arm portion including one end portion closest to the main body and an opposite end portion remote from the main body, the opposite end portion of the first arm portion including an enlarged portion that protrudes in a width direction of the first arm portion, the second arm portion being longer than the first arm portion;
wrapping the first arm portion and the second arm portion around a limb of the patient while the main body is in overlying relation to the puncture site on the hand of the patient;
securing a first part of the second arm portion and the enlarged portion of the first arm to one another while the first part of the second arm portion is in contact with the enlarged portion of the first arm;
passing the second arm portion between two fingers of the hand of the patient;
securing a second part of the second arm portion and a third part of the second arm portion to one another while the second part of the second arm portion and the third part of the second arm portion are in contact with one another; and
introducing a fluid into the inflatable member to inflate the inflatable member and apply a compressive force from the inflatable member to the puncture site to perform hemostasis.

19. The method according to claim 18, wherein the securing of the second part of the second arm portion and the third part of the second arm portion to one another occurs after the securing of the first part of the second arm portion and the enlarged portion of the first arm.

20. The method according to claim 18, wherein the passing of the second arm portion between the two fingers of the hand of the patient occurs before the securing of the second part of the second arm portion and the third part of the second arm portion to one another.
